(19) United States
(12) Patent Application Publication
(10) Pub. No.: US 2005/0038047 A1
(43) Pub. Date: Feb. 17, 2005

(54) Azaquinazoline derivatives

(76) Inventors: Paul John Edwards, Sandwich (GB); Karl Richard Gibson, Sandwich (GB); Simon John Mantell, Sandwich (GB); Graham Nigel Maw, Sandwich (GB); Cedric Poinsard, Sandwich (GB)

Correspondence Address: WARNER-LAMBERT COMPANY, 2800 PLYMOUTH RD, ANN ARBOR, MI, 48105, US

(21) Appl. No.: 10/917,690

(22) Filed: Aug. 13, 2004

Related U.S. Application Data
(60) Provisional application No. 60/508,410, filed on Oct. 2, 2003.
(60) Provisional application No. 60/560,515, filed on Apr. 7, 2004.

(30) Foreign Application Priority Data
Aug. 14, 2003 (GB) 0319148.3
Mar. 16, 2004 (GB) 0405905.1

(51) Int. Cl. A61K 31/519; C07D 487/02

(52) U.S. Cl. 514/264.11; 544/280

(57) ABSTRACT

The present invention relates to certain novel 6-amino-7-azaquinazoline derivatives and to processes for the preparation of, intermediates used in the preparation of, compositions containing and the uses of, such derivatives. One important use is in the treatment of pain.

This application is a United States utility application, which claims the benefit of priority to United Kingdom patent application Serial No. 0319148.3 filed Aug. 14, 2003, U.S. provisional application No. 60/508,410, filed Oct. 2, 2003, United Kingdom patent application Serial No. 0405905.1 filed Mar. 16, 2004, and U.S. provisional application No. 60/560,515, filed Apr. 7, 2004.

This invention relates to azaquinazoline derivatives. More particularly, this invention relates to 6-amino-7-azaquinazoline derivatives and to processes for the preparation of, intermediates used in the preparation of, compositions containing and the uses of, such derivatives.

The azaquinazoline derivatives of the present invention are antagonists of the human metabotropic glutamate subtype 1 receptor (mGluR1) and have a number of therapeutic applications, particularly in the treatment of pain.

The azaquinazoline derivatives of the invention are selective mGluR1 receptor antagonists. In particular, they show an affinity for the mGluR1 receptor which is greater than their affinity for the mGluR5 receptor. Preferred compounds of the invention show at least a 100-fold selectivity for the mGluR1 receptor as compared with the mGluR5 receptor. Advantageously, the compounds of the invention show little or no affinity for the enzyme EGFR kinase.

Glutamate is an important neurotransmitter in the central nervous system, activating cation channels via ionotropic glutamate receptors and modulating cell excitability by action at G-protein coupled metabotropic glutamate receptors (mGluRs). Eight metabotropic glutamate receptor subtypes have been identified and further classified into three distinct groups based on their sequence homology, pharmacology and coupling to intracellular effector mechanisms. The group I mGluRs, consisting of mGluR1 and mGluR5, are postsynaptic and are primarily coupled to phospholipase C, regulating neuronal excitability via phosphoinositide hydrolysis, release of intracellular Ca²⁺ and activation of protein kinase C. Group II (mGluR2, mGluR3) and group III (mGluR4, mGluR6, mGluR7, mGluR8) are primarily presynaptic auto receptors and inhibit adenylyl cyclase through a pertusis toxin sensitive G-protein, inhibiting cyclic AMP formation and the activation of cAMP dependent protein kinase.

Activity at excitatory glutamatergic pathways is associated with a number of neuropathologies including: ischaemic damage, neurodegeneration, pain, depression, drug dependency, epilepsy, Parkinsonism and schizophrenia. Group 1 metabotropic glutamate receptors are localized in brain regions primarily associated with somatosensory processing, including the dorsal root ganglia, superficial and deep spinal dorsal horn, midbrain reticular formation, somatosensory thalamus, amygdala and cortex. Metabotropic glutamate receptors, particularly mGluR1, have been shown to be involved in processes of nociception and hyperalgesia. Antibodies raised against mGluR1 increase tail flick latencies and reduce the responses of dorsal horn neurones to repeated noxious stimuli (Young et al, Journal of Neuroscience, 18, 10180-10188, 1998). Intrathecal group I agonists cause heat hyperalgesia and increase nociceptive behaviours (Fisher and Coderre, Neuroreport, 9, 1169-1172, 1998). There is evidence to suggest that group I mGluRs are involved in more persistent pain conditions since administration of specific mGluR1 antibodies reduced cold allodynia following nerve damage (Fundytus et al., Neuroreport, 9, 731-5, 1998). Antisense oligonucleotides demonstrate that ablation of the mGluR1 receptor attenuates the hyperalgesia and allodynia associated with nerve damage (Fundytus et al, British Journal of Pharmacology, 132, 354-67, 2001) and attenuates the hyperalgesia and allodynia that occurs as a result of chronic inflammation (Fundytus et al, Pharmacology, Biology and Behaviour, 73, 401-10, 2002). Spinal administration of a group I receptor antagonist will reverse the central sensitisation induced by peripheral capsaicin injection (Neugebauer et al, Journal of Neurophysiology, 82, 272-82, 1999). Group I receptor agonists enhance behavioural responses and group I receptor antagonists attenuate behavioural responses in the late phase of the formalin test (Fisher and Coderre, Pain, 68, 255-263, 1996).

Cerebral ischemia causes an increased release of glutamate and an increase in intracellular calcium which can cause cell death and neuronal degeneration. Compounds, which regulate activity at mGluR1, are neuroprotective in animal models of cerebral ischemia (De Vry et al, European Journal of Pharmacology, 428, 203-14, 2001).

Hyper-excitability in the central nervous system (CNS) can result in seizures and convulsions, apoptosis and neurodegeneration. mGluR1 receptor antagonists have been shown to inhibit seizures in animal models indicating a likely utility in epilepsy (Chapman et al, European Journal of Pharmacology, 368, 17-24, 1999). In developing animals, kainic acid administration causes recurrent seizures and hippocampal dysfunction impairing learning ability. Group I receptor antagonists are both neuroprotective and also improve learning performance in this model (Renaud et al, Epilepsia, 43, 1306-1317, 2002) indicating a possible application in the treatment of neurodegenerative disease states such as Alzheimers and dementia (including HIV induced dementia).

The compounds of the present invention are therefore potentially useful in the treatment of a wide range of disorders, particularly in the treatment of pain, epilepsy and neurodegenerative disorders such as Alzheimer's disease, dementia and stroke.

The treatment of pain, particularly nociceptive pain, is a preferred use. Physiological pain is an important protective mechanism designed to warn of danger from potentially injurious stimuli from the external environment. The system operates through a specific set of primary sensory neurones and is exclusively activated by noxious stimuli via peripheral transducing mechanisms (see Millan, 1999, Prog. Neurobio., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly or via brain stem relay nuclei, to the ventrobasal thalamus and then on to the cortex, where the sensation of pain is generated.

Intense acute pain and chronic pain often involve the same pathways but driven by pathophysiological processes and as such ceasing to provide a protective mechanism and instead contributing to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful, allowing for repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter, 2000, Science, 288, 1765-1768). Clinical pain is present when discomfort and abnormal sensitivity feature among the patient's symptoms. Patients tend to be quite heterogeneous and may present with various pain symptoms. There are a number of typical pain subtypes including: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients with back pain, arthritic pain, CNS trauma, or neuropathic pain may have similar symptoms, the underlying mechanisms are different and, therefore, may require different treatment strategies. Therefore pain can be divided into a number of different areas, because of differing pathophysiology, including nociceptive, inflammatory and neuropathic pain. It should be noted that some types of pain have multiple aetiologies and thus can be classified in more than one area, e.g. back pain and cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, renal colic, cancer pain (which may be tumour related pain, e.g. bone pain, headache, facial pain and visceral pain, or associated with cancer therapy, e.g. postchemotherapy syndromes, chronic postsurgical pain syndromes, and post radiation syndromes) and back pain (which may be due to herniated or ruptured intervertabral discs or abnormalities of the lumber facet joints, sacroiliac joints, paraspinal muscles or the posterior longitudinal ligament).

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, diabetic neuropathy, post herpetic neuralgia, back pain, cancer neuropathy, HIV neuropathy, phantom limb pain, carpal tunnel syndrome, chronic alcoholism, hypothyroidism, trigeminal neuralgia, uremia and vitamin deficiencies. Neuropathic pain is pathological as it has no protective role. It is often present well after the original cause has dissipated, commonly lasting for years, significantly decreasing a patients quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Another type of inflammatory pain is the pain associated with inflammatory bowel disease (IBD).

Other types of pain include but are not limited to:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
central pain or ‘thalamic pain’, as defined by pain caused by lesion or dysfunction of the nervous system, including central post-stroke pain, multiple sclerosis, spinal cord injury, Parkinson's disease and epilepsy;
heart and vascular pain, including angina, myocardical infarction, mitral stenosis, pericarditis, Raynaud's phenomenon, scleredoma and skeletal muscle ischemia;
visceral pain and gastrointestinal disorders;
head pain, such as migraine (including migraine with aura and migraine without aura), cluster headache and tension-type headache; and
orofacial pain, including dental pain and temporomandibular myofascial pain.

The viscera encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, which all regularly produce visceral pain. Other types of visceral pain include the pain associated with dysmenorrhea, pelvic pain, cystitis and pancreatitis.

The compounds of the present invention are potentially useful in the treatment of all kinds of pain, particularly nociceptive pain.

Other specific conditions that may be treated with the compounds of the present invention include Alzheimer's disease, amyotrophic lateral sclerosis, anxiety, brain edema, cerebral deficits subsequent to cardiac bypass surgery and grafting, cerebral ischaemia, cognitive disorders, convulsions, dementia (including HIV-induced dementia), depression, drug intoxication, drug-tolerance and withdrawal, emesis, epilepsy, head trauma, Huntington's chorea, hypoglycaemic neuronal damage, inherited ataxias, metabolic derangement, muscular spasms, ocular damage, Parkinson's disease, perinatal hypoxia, psychosis including schizophrenia and bipolar disorder), post-traumatic stress disorder, retinopathy, spasticity, spinal cord lesions due to trauma or infarction/ischaemia or inflamation, stroke, tardive dyskinesia, tremor and urinary incontinence.

There is a need to provide new mGluR1 receptor antagonists that are better drug candidates. In particular, such compounds should bind potently to the mGluR1 receptor whilst showing little affinity for other receptors and show functional activity as antagonists. They should be well absorbed from the gastrointestinal tract, be relatively metabolically stable and possess favourable pharmacokinetic properties. When targeted selectively against receptors in the central nervous system they should cross the blood brain barrier freely and when targeted selectively against receptors in the peripheral nervous system they should not cross the blood brain barrier. They should be non-toxic and demonstrate few side-effects. Furthermore, the ideal drug candidate will exist in a physical form that is stable, non-hygroscopic and easily formulated.

The invention provides a compound of formula (I): 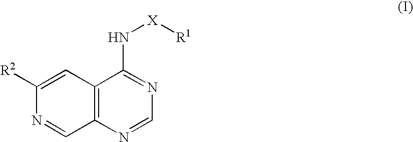
or a pharmaceutically acceptable salt or solvate thereof, wherein
X is a bond or C₁-C₃ alkylene;
R¹ is (a) C₃-C₈ cycloalkyl optionally substituted with one or more substituents selected from halo, oxo, C₁-C₆ alkyl, C₃-C₈ cycloalkyl, Het¹, C₁-C₆ alkoxy and cyano, wherein one or two of the methylene (—CH₂—) groups of said C₃-C₈ cycloalkyl may optionally be replaced by an —NR³—, —O— or —S(O)ₙ— group and wherein said C₃-C₈ cycloalkyl, whether modified as indicated above or not, may be optionally benzo-fused, said benzo-fused portion being optionally substituted by one or more substituents selected from halo, C₁-C₆ alkyl, C₃-C₈ cycloalkyl, C₁-C₆ alkoxy and cyano; or
   (b) C₃-C₈ cycloalkyl spiro fused to a C₃-C₈ cycloalkyl or Het¹ group; or
   (c) Het²;
   with the proviso that R¹ may not be Het² when X is a bond;
R² is —OR⁴ or —NR⁴R⁵;
R³ is H, C₁-C₆ alkyl or C₃-C₈ cycloalkyl;
R⁴ is C₁-C₆ alkyl or C₃-C₈ cycloalkyl, said C₁-C₆ alkyl and C₃-C₈ cycloalkyl being optionally substituted by one or more R⁶ or —(C₁-C₆ alkylene)-R⁶ groups and optionally having one methylene group (—CH₂—) replaced by an —NR³—, —O— or —S(O)ₙ— group and said C₃-C₈ cycloalkyl, whether modified as indicated above or not, being optionally benzo-fused, said benzo-fused portion being optionally substituted by one or more substituents selected from halo, C₁-C₆ alkyl, C₃-C₈ cycloalkyl, C₁-C₆ alkoxy and cyano; and
R⁵ is H, C₁-C₆ alkyl or C₃-C₈ cycloalkyl;
or, in the case where R² is —NR⁴R⁵, R⁴ and R⁵, taken together, with the nitrogen atom to which they are attached, form a saturated heterocyclic group selected from aziridinyl, azetidinyl, pyrrolidinyl, piperidinyl, morpholinyl, thiomorpholinyl, piperazinyl, azepinyl or diazapinyl, wherein said heterocyclic group is optionally substituted on a ring carbon atom by one or more R⁶ or —(C₁-C₆ alkylene)—R⁶ groups, optionally substituted on a ring nitrogen atom by one or more R⁹ groups and optionally benzo-fused, said benzo-fused portion being optionally substituted by one or more substituents selected from halo, C₁-C₆ alkyl, C₃-C₈ cycloalkyl, C₁-C₆ alkoxy and cyano;
R⁶ is Het¹, Het², —OR⁷, —SR⁷, —SOR⁸, —SO₂R⁸, —NR⁷R⁷, —COR⁷, —OCOR⁷, —SCOR⁷,
—NR⁷COR⁷, —NR⁷SO₂R⁸, —COOR⁷, —COSR⁷, —CONR⁷R⁷, —OCOOR⁸—OCOSR⁸,
—OCONR⁷R⁷, —NR⁷COOR⁷, —NR⁷COSR⁷, —NR⁷CONR⁷R⁷, oxo, halo, —CN, C₁-C₆ alkyl, C₃-C₈ cycloalkyl, C₂-C₆ alkenyl, C₂-C₆ alkynyl or aryl;
each R⁷ is independently selected from H, C₁-C₆ alkyl and C₃-C₈ cycloalkyl;
each R⁸ is independently selected from C₁-C₆ alkyl and C₃-C₈ cycloalkyl;
R⁹ is C-linked Het¹, C-linked Het², —SO₂R⁸, —COR⁷, —COOR⁸, —COSR⁸, —CONR⁷R⁷, C₁-C₆ alkyl, C₃-C₈ cycloalkyl, C₂-C₆ alkenyl, C₂-C₆ alkynyl or aryl;
n is 0, 1 or 2;
Het¹ is a 3- to 8-membered, saturated or partially unsaturated heterocyclic group comprising one or two ring members selected from —NR¹⁰—, —O— and —S(O)ₙ—, said heterocyclic group being optionally substituted on a ring carbon atom by one or more substituents selected from oxo, halo, —R⁸ or —OR⁸ and optionally benzo-fused, said benzo-fused portion being optionally substituted by one or more substituents selected from halo, C₁-C₆ alkyl, C₃-C₈ cycloalkyl, C₁-C₆ alkoxy and cyano;
R¹⁰ is H, C₁-C₆ alkyl or C₃-C₈ cycloalkyl, —COR⁸, —SO₂R⁸ or a bond to the group which is substituted with Het¹;
Het² is a 5-membered aromatic heterocyclic group comprising either (a) 1 to 4 nitrogen atoms, (b) one oxygen or one sulphur atom or (c) 1 oxygen atom or 1 sulphur atom and 1 or 2 nitrogen atoms or a 6-membered aromatic heterocyclic group comprising 1 or 2 nitrogen atoms, said 5- or 6-membered heterocyclic group being optionally substituted by one or more substituents selected from halo, —NR⁷R⁷, C₁-C₆ alkyl, C₃-C₈ cycloalkyl, C₁-C₆ alkoxy and cyano and optionally benzo-fused, said benzo-fused portion being optionally substituted by one or more substituents selected from halo, —NR⁷R⁷, C₁-C₆ alkyl, C₃-C₈ cycloalkyl, C₁-C₆ alkoxy and cyano; and
aryl is phenyl or naphthyl optionally substituted by one or more substituents selected from halo, —NR⁷R⁷, C₁-C₆ alkyl, C₃-C₈ cycloalkyl, C₁-C₆ alkoxy and cyano.

In the above definitions, halo means fluoro, chloro, bromo or iodo and alkyl, alkenyl, alkynyl, alkylene, and alkoxy groups containing the requisite number of carbon atoms can be unbranched or branched chain. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy. Examples of alkylene include methylene, 1,1-ethylene, 1,2-ethylene, 1,1-propylene, 1,2-propylene, 1,3-propylene and 2,2-propylene. Examples of cycloalkyl include cyclopropyl, cyclobutyl, cyclopentyl, cyclohexyl and cycloheptyl. The term ‘C-linked’, used in the definition of R⁹, means that the Het¹ or Het² group is joined via a ring carbon atom. In the definition of R¹, ‘spiro’ fusion of a C₃-C₈ cycloalkyl group to a C₃-C₈ cycloalkyl or Het¹ group means that the two C₃-C₈ cycloalkyl groups or the C₃-C₈ cycloalkyl and Het¹ group share a carbon atom in common. Thus, for instance, where R¹ is cyclopentyl spiro fused to cyclobutyl, the R¹ group as a whole is spiro[3.4]octanyl.

Specific examples of Het¹ are oxiranyl, aziridinyl, oxetanyl, azetidinyl, tetrahydrofuranyl, pyrrolidinyl, tetrahydropyranyl, piperidinyl, morpholinyl, thiomorpholinyl, piperazinyl, azepinyl, oxapinyl, 1,4-oxazepinyl and 1,4-diazepinyl (optionally substituted and benzo-fused as specified above).

Specific examples of Het² are thienyl, furanyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyrimidinyl, pyrazinyl and pyridazinyl (optionally substituted and benzo-fused as specified above).

Preferably, Het¹ is a 3- to 6-membered, saturated heterocyclic group comprising one or two ring members selected from —NR⁸— and —O—, said heterocyclic group being optionally substituted on a ring carbon atom by one or more substituents selected from oxo, halo, —R⁸ or —OR⁸ and optionally benzo-fused, said benzo-fused portion being optionally substituted by one or more substituents selected from halo, C₁-C₆ alkyl, C₃-C₈ cycloalkyl, C₁-C₆ alkoxy and cyano.

More preferably, Het¹ is a 3- to 6-membered, saturated heterocyclic group comprising one or two ring members selected from —NR⁸— and —O—, said heterocyclic group being optionally substituted on a ring carbon atom by one or more oxo groups.

Most preferably, Het¹ is aziridinyl, tetrahydrofuranyl, piperidinyl, morpholinyl, pyrrolidinyl or piperazinyl, each optionally substituted on a ring carbon atom by one or more oxo groups and optionally substituted on a ring nitrogen atom by C₁-C₆ alkyl.

In one embodiment, Het² is a 5-membered aromatic heterocyclic group comprising either (a) 1 to 4 nitrogen atoms, (b) one oxygen or one sulphur atom or (c) 1 oxygen atom or 1 sulphur atom and 1 or 2 nitrogen atoms or a 6-membered aromatic heterocyclic group comprising 1 or 2 nitrogen atoms, said 5- or 6-membered heterocyclic group being optionally substituted by one or more substituents selected from halo, —NR⁷R⁷, C₁-C₆ alkyl, C₁-C₆ alkoxy and cyano and optionally benzo-fused, said benzo-fused portion being optionally substituted by one or more substituents selected from halo, —NR⁷R⁷, C₁-C₆ alkyl, C₃-C₈ cycloalkyl, C₁-C₆ alkoxy and cyano.

Preferably, Het² is a 5-membered aromatic heterocyclic group comprising (a) 2 to 4 nitrogen atoms or (b) one oxygen atom and 1 or 2 nitrogen atoms or a 6-membered aromatic heterocyclic group comprising 1 or 2 nitrogen atoms, said 5- or 6-membered heterocyclic group being optionally substituted by one or more substituents selected from halo, C₁-C₆ alkyl, C₃-C₈ cycloalkyl, C₁-C₆ alkoxy and cyano and optionally benzo-fused, said benzo-fused portion being optionally substituted by one or more substituents selected from halo, —NR⁷R⁷, C₁-C₆ alkyl, C₃-C₈ cycloalkyl, C₁-C₆ alkoxy and cyano.

More preferably, Het² is a 5-membered aromatic heterocyclic group comprising (a) 2 to 4 nitrogen atoms or (b) one oxygen atom and 1 or 2 nitrogen atoms or a 6-membered aromatic heterocyclic group comprising 1 or 2 nitrogen atoms, said 5- or 6-membered heterocyclic group being optionally substituted by one or more substituents selected from halo, —NR⁷R⁷, C₁-C₆ alkyl, C₃-C₈ cycloalkyl, C₁-C₆ alkoxy and cyano.

More preferably still, Het² is a heterocyclic group selected from imidazolyl, pyrazolyl, triazolyl, oxazolyl, pyridyl and pyrimidinyl, said heterocyclic group being optionally substituted by one or more substituents selected from halo, —NR⁷R⁷, C₁-C₆ alkyl, C₃-C₈ cycloalkyl, C₁-C₆ alkoxy and cyano.

Most preferably, Het² is imidazolyl or pyridyl said imidazolyl and pyridyl being optionally substituted by C₁-C₆ alkyl.

Preferably, R⁶ is Het¹, Het², —OR⁷, —NR⁷R⁷, —COR⁷, —NR⁷COR⁷, —NR⁷SO₂R⁸, —COOR⁷, ——CONR⁷R⁷, oxo, halo, C₁-C₆ alkyl, C₃-C₈ cycloalkyl or aryl;

Most preferably, R⁶ is —NR⁷SO₂R⁸, Het¹, Het², halo, —NR⁷R⁷ or —OR⁷.

Preferably, R⁹ is —COR⁷.

In a preferred aspect (A), the invention provides a compound of formula (I), or a pharmaceutically acceptable salt or solvate thereof, wherein R¹ and R² are as defined above, and X is
(a) methylene or a bond; or
(b) methylene; or
(c) a bond.

In a preferred aspect (B), the invention provides a compound of formula (I), or a pharmaceutically acceptable salt or solvate thereof, wherein R² is as defined above, X is as defined above, either in its broadest aspect or in a preferred aspect under (A)(a)-(c) and R¹ is:
(a) (i) C₅-C₈ cycloalkyl optionally substituted with one or more substituents selected from halo, oxo, C₁-C₆ alkyl, C₃-C₈ cycloalkyl, Het¹, C₁-C₆ alkoxy and cyano, wherein one or two of the methylene (—CH₂—) groups of said C₅-C₈ cycloalkyl may optionally be replaced by an —NR³—, —O— or —S(O)ₙ— group and wherein said C₅-C₈ cycloalkyl, whether modified as indicated above or not, may be optionally benzo-fused, said benzo-fused portion being optionally substituted by one or more substituents selected from halo, C₁-C₆ alkyl, C₃-C₈ cycloalkyl, C₁-C₆ alkoxy and cyano; or (ii) Het²; with the proviso that R¹ may not be Het² when X is a bond; or
(b) (i) C₅-C₈ cycloalkyl optionally substituted with one or more substituents selected from halo and C₁-C₆ alkyl, wherein one of the methylene (—CH₂—) groups of said C₅-C₈ cycloalkyl may optionally be replaced by an —O— group and wherein said C₅-C₈ cycloalkyl, whether modified as indicated above or not, may be optionally benzo-fused; or (ii) pyridyl optionally substituted by one or more C₁-C₆ alkyl groups; with the proviso that R¹ may not be optionally substituted pyridyl when X is a bond; or
(c) cyclohexyl, cycloheptyl, cyclooctyl, methylcyclohexyl, dimethylcyclohexyl, difluorocyclohexyl, tetrahydrofuranyl, indanyl, pyridyl or methylpyridyl; or
(d) cyclohexyl, cycloheptyl, cyclooctyl, 4-methylcyclohexyl, 4,4-dimethylcyclohexyl, 4,4-difluorocyclohexyl, tetrahydrofuran-2-yl, indan-2-yl, pyrid-2-yl or 6-methylpyrid-2-yl.

In a preferred aspect (C), the invention provides a compound of formula (I), or a pharmaceutically acceptable salt or solvate thereof, wherein R² is as defined above and —X—R¹ is:
(a) cyclohexyl, cycloheptyl, cyclooctyl, methylcyclohexyl, dimethylcyclohexyl, difluorocyclohexyl, tetrahydrofuranylmethyl, indanyl, pyridylmethyl or methylpyridylmethyl; or
(b) cyclohexyl, cycloheptyl, cyclooctyl, 4-methylcyclohexyl, 4,4-dimethylcyclohexyl, 4,4-difluorocyclohexyl, tetrahydrofuran-2-ylmethyl, indan-2-yl, pyrid-2-ylmethyl or (6-methylpyrid-2-yl)methyl.

In a preferred aspect (D), the invention provides a compound of formula (I), or a pharmaceutically acceptable salt or solvate thereof, wherein X is as defined above, either in its broadest aspect or in a preferred aspect under (A)(a)-(c) and R¹ is as defined above, either in its broadest aspect or in a preferred aspect under (B)(a)-(d), or —X—R¹ is as defined above under (C)(a)-(b) and R² is —OR⁴ or —NR⁴R⁵ wherein:
(a) R⁴ is C₁-C₅ alkyl or C₅-C₆ cycloalkyl, said C₁-C₅ alkyl and C₅-C₆ cycloalkyl being optionally substituted by one or more R⁶ or —(C₁-C₆ alkylene)-R⁶ groups and optionally having one methylene (—CH₂—) group replaced by an —NR³—, —O— or —S(O)ₙ— group and said C₅-C₆ cycloalkyl, whether modified as indicated above or not, being optionally benzo-fused, said benzo-fused portion being optionally substituted by one or more substituents selected from halo, C₁-C₆ alkyl, C₃-C₈ cycloalkyl, C₁-C₆ alkoxy and cyano and R⁵ is H; or in the case where R² is —NR⁴R⁵, R⁴ and R⁵ when taken together with the nitrogen atom to which they are attached, form a saturated heterocylic group selected from azetidinyl, piperidinyl and piperazinyl, wherein said heterocyclic group is optionally substituted on a ring carbon atom by one or more R⁶ or —(C₁-C₆ alkylene)-R⁶ groups, optionally substituted on a ring nitrogen atom by one or more R⁹ groups and optionally benzo-fused, said benzo-fused portion being optionally substituted by one or more substituents selected from halo, C₁-C₆ alkyl, C₃-C₈ cycloalkyl C₁-C₆ alkoxy and cyano; or
(b) R⁴ is C₁-C₅ alkyl or C₅-C₆ cycloalkyl, said C₁-C₅ alkyl and C₅-C₆ cycloalkyl being optionally substituted by one or more groups selected from Het¹, Het², —OR⁷—NR⁷R⁷ and —NR⁷SO₂R⁷ and optionally having one methylene (—CH₂—) group replaced by an —O-group and said C₅-C₆ cycloalkyl, whether modified as indicated above or not, being optionally benzo-fused and R⁵ is H; or in the case where R² is —NR⁴R⁵, R⁴ and R⁵ when taken together with the nitrogen atom to which they are attached, form a saturated heterocylic group selected from azetidinyl, piperdinyl and piperazinyl, wherein said heterocyclic group is optionally substituted on a ring carbon atom by one ore more groups selected from —OR⁷—(C₁-C₆ alkylene)-NR⁷R⁷ and halo and optionally substituted on a ring nitrogen atom by —COR⁷; or
(c) R⁴ is tetrahydrofuranylmethyl, methylsulphonamidoethyl, methoxyethyl, (N-methylpyrrolidinyl)ethyl, morpholinylethyl, hydroxypropyl, N,N-dimethylaminopropyl, pyrrolidinylpropyl, morpholinylpropyl, imidazolylpropyl, (N,N-dimethylamino)butyl, (hydroxyethyloxy)ethyl, indanyl, cyclohexyl, pyrrolidinonylmethyl, N-methylpiperidinylmethyl, azetidinylethyl, pyrrolidinonylethyl, (1-methylpiperazinyl)ethyl, (1-methylpiperazinyl)propyl or hydroxycyclohexyl and R⁵ is H; or in the case where R² is —NR⁴R⁵, R⁴ and R⁵ when taken together with the nitrogen atom to which they are attached, form an azetidinyl, (N,N-dimethylaminomethyl)piperidinyl, acetylpiperazinyl, hydroxypiperidinyl, methoxypiperidinyl or difluoropiperidinyl group; or
(d) R⁴ is tetrahydrofuran-2-ylmethyl, 2-(methylsulphonamido)ethyl, 2-methoxyethyl, 2-(N-methylpyrrolidin-2-yl)ethyl, 2-(morpholin-4-yl)ethyl, 3-hydroxypropyl, 3-(N,N-dimethylamino)propyl, 3-(pyrrolidin-1-yl)propyl, 3-(morpholin-4-yl)propyl, 3-(imidazol-1-yl)propyl, 4-(N,N-dimethylamino)butyl, 2-((2-hydroxyethyl)oxy)ethyl, indan-2-yl, cyclohexyl, tetrahydrofuran-3-ylmethyl, (pyrrolidin-2-on-5-yl)methyl, (N-methylpiperidin-4-yl)methyl, 2-azetidinylethyl, 2-(pyrrolidin-2-on-1-yl)ethyl, 2-(1-methylpiperazin-4-yl)ethyl, 3-(1-methylpiperazin-4-yl)propyl or 4-hydroxycyclohexyl and R⁵ is H; or in the case where R² is —NR⁴R⁵, R⁴ and R⁵ when taken together with the nitrogen atom to which they are attached, form an azetidinyl, 4-(N,N-dimethylaminomethyl)piperidin-1-yl, 4-acetylpiperazin-1-yl, 4-hydroxypiperidin-1-yl, 4-methoxypiperidin-1-yl or 4,4-difluoropiperidin-1-yl group.

In a preferred aspect (E), the invention provides a compound of formula (I), or a pharmaceutically acceptable salt or solvate thereof, wherein X is as defined above, either in its broadest aspect or in a preferred aspect under (A)(a)-(c) and R¹ is as defined above, either in its broadest aspect or in a preferred aspect under (B)(a)-(d), or —X—R¹ is as defined above under (C)(a)-(b) and R² is:
(a) (tetrahydrofuranylmethyl)amino, (methylsulphonamidoethyl)amino, (methoxyethyl)amino, ((N-methylpyrrolidinyl)ethyl)amino, (morpholinylethyl)amino, (hydroxypropyl)amino, (N,N-dimethylaminopropyl)amino, (pyrrolidinylpropyl)amino, (morpholinylpropyl)amino, (imidazolylpropyl)amino, ((N,N-dimethylamino)butyl)amino, ((hydroxyethyloxy)ethyl)amino, indanylamino, cyclohexylamino, (tetrahydrofuranylmethyl)oxy, (pyrrolidinonylmethyl)oxy, (N-methylpiperidinylmethyl)oxy, (azetidinylethyl)oxy, (pyrrolidinonylethyl)oxy, (morpholinylethyl)oxy, ((1-methylpiperazinyl)ethyl)oxy, (N,N-dimethylaminopropyl)oxy, ((1-methylpiperazinyl)propyl)oxy, (hydroxycyclohexyl)oxy, azetidinyl, (N,N-dimethylaminomethyl)piperidinyl, acetylpiperazinyl, hydroxypiperidinyl, methoxypiperidinyl or difluoropiperidinyl; or
(b) (tetrahydrofuran-2-ylmethyl)amino, (2-(methylsulphonamido)ethyl)amino, (2-methoxyethyl)amino, (2-(N-methylpyrrolidin-2-yl)ethyl)amino, (2-(morpholin-4-yl)ethyl)amino, (3-hydroxypropyl)amino, (3-(N,N-dimethylamino)propyl)amino, (3-(pyrrolidin-1-yl)propyl)amino, (3-(morpholin-4-yl)propyl)amino, (3-(imidazol-1-yl)propyl)amino, (4-(N,N-dimethylamino)butyl)amino, (2-((2-hydroxyethyl)oxy)ethyl)amino, (indan-2-yl)amino, cyclohexylamino, (tetrahydrofuran-3-ylmethyl)oxy, ((pyrrolidin-2-on-5-yl)methyl)oxy, ((N-methylpiperidin-4-yl)methyl)oxy, (2-azetidinylethyl)oxy, (2-(pyrrolidin-2-on-1-yl)ethyl)oxy, (2-(morpholin-4-yl)ethyl)oxy, (2-(1-methylpiperazin-4-yl)ethyl)oxy, (3-(N,N-dimethylamino)propyl)oxy, (3-(1-methylpiperazin-4-yl)propyl)oxy or (4-hydroxycyclohexyl)oxy, azetidinyl, 4-(N,N-dimethylaminomethyl)piperidin-1-yl, 4-acetylpiperazin-1-yl, 4-hydroxypiperidin-1-yl, 4-methoxypiperidin-1-yl or 4,4-difluoropiperidin-1-yl.

Specific preferred compounds of formula (I) are those listed in the Examples section below and the pharmaceutically acceptable salts and solvates thereof, particularly Examples 14, 24 and 27 and the pharmaceutically acceptable salts and solvates thereof.

Pharmaceutically acceptable salts of a compound of formula (I) include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, Weinheim, Germany, 2002).

Pharmaceutically acceptable salts of a compound of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

A compound of formula (I) may exist in both unsolvated and solvated forms. The term ‘solvate’ is used herein to describe a molecular complex comprising the compound of formula (I) and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J. Pharm. Sci., 64 (8), 1269-1288, by Haleblian (August 1975).

Hereinafter, all references to a compound of formula (I) include references to salts, solvates and complexes thereof and to solvates and complexes of salts thereof.

A compound of formula (I), as hereinbefore defined, may exist in one or more crystalline (polymorphic) or isomeric forms (including optical, geometric and tautomeric isomers), in an isotopically labelled form or as a prodrug. All such crystalline/isomeric forms and prodrugs are within the scope of the present invention and are further described below. All references to a compound of formula (I) should be interpreted accordingly.

As indicated, so-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include
(i) where the compound of formula (I) contains a carboxylic acid functionality (—COOH), an ester thereof, for example, a compound wherein the hydrogen of the carboxylic acid functionality of the compound of formula (I) is replaced by (C₁-C₈)alkyl;
(ii) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(iii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃—>—CH₂OH):
(ii) where the compound of formula (I) contains an alkoxy group, an hydroxy derivative thereof (—OR—>—OH);
(iii) where the compound of formula (I) contains a tertiary amino group, a secondary amino derivative thereof (—NR¹R²—>—NHR¹ or —NHR²);
(iv) where the compound of formula (I) contains a secondary amino group, a primary derivative thereof (—NHR¹—>—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂—>COOH).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art—see, for example, Stereochemistry of Organic Compounds by E. L. Eliel and S. H. Wilen (Wiley, New York, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, ie. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

All of the compounds of formula (I) can be prepared by conventional routes such as by the procedures described in the general methods presented below or by the specific methods described in the Examples section and the Preparations section, or by similar methods thereto. The present invention also encompasses any one or more of these processes for preparing the compounds of formula (I), in addition to any novel intermediates used therein.

In the following general methods, R¹, R², R⁴, R⁵ and X are as previously defined for a compound of formula (I) unless otherwise stated.

A compound of formula (I) may be prepared by the reaction of a compound of the formula (II) 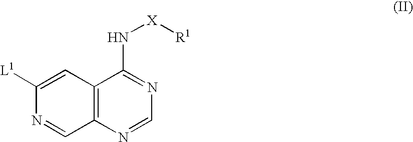
wherein L¹ is a suitable leaving group, with a compound of the formula
R⁴OH (III)
or a compound of the formula
R⁴R⁵NH (IV).

L¹ is preferably halo, most preferably fluoro. The reaction is optionally carried out in a suitable solvent, such as dichloromethane or dimethylsulphoxide. The temperature of the reaction may optionally be elevated, preferably to from 80° C. to 130° C., where no solvent or a sufficiently high-boiling solvent such as dimethylsulphoxide is used. The use of microwave radiation is particularly effective.

A compound of the formula (II) may be prepared by the reaction of a compound of the formula (V) 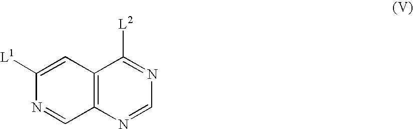
wherein L² is a suitable leaving group, with a compound of the formula
R¹—X—NH₂ (VI).

L² is preferably halo, most preferably chloro. Preferably, a base is added to the reaction mixture. Where an amine base, such as N-ethyl-N,N-diisopropylamine, triethylamine, N-methylmorpholine or aminopyridine, is chosen, the reaction is preferably carried out in a suitable solvent, such as dichloromethane, N,N-dimethylformamide or dimethylsulphoxide, at a temperature at or above room temperature. Where an alkali metal base, such as butyllithium or sodium hexamethyldisilazide, is chosen, the reaction is preferably carried out in a suitable solvent, such as tetrahydrofuran or dioxan, at a temperature below room temperature. Most preferably, the reaction is carried out in the presence of a tertiary amine base, as a solution in dichloromethane, at room temperature.

Compounds of formula (I) can also be prepared by using the reactions described above to construct a compound wherein R¹ or R² are partially formed and then completing the synthesis by functional group manipulation. For instance, where R¹ or R² bears a hydroxy or amino group, this group may optionally be carried through the synthesis in a protected form and deprotected in a final step. Suitable protecting groups are described in ‘Protective Groups in Organic Synthesis’ by Theorora Greene and Peter Wuts (third edition, 1999, John Wiley and Sons). Equally where R¹ or R² includes an oxygen, nitrogen or other nucleophilic atom, the final step in the synthesis may consist of functionalising a hydroxy or amino group, for instance by the formation of a sulphonamide (see Example 10). Suitable functional group transformations are described in ‘Comprehensive Organic Transformations’ by Richard Larock (1999, VCH Publishers Inc.).

Compounds of formula (I) may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of formula (I) or in combination with one or more other drugs (or as any combination thereof. Generally, they will be administered as a formulation in association with one or more pharmaceutically acceptable excipients. The term ‘excipient’ is used herein to describe any ingredient other than a compound of formula (I). The choice of excipient will to a large extent depend on factors such as the particular mode of administration, the effect of the excipient on solubility and stability, and the nature of the dosage form.

Pharmaceutical compositions suitable for the delivery of compounds of formula (I) and methods for their preparation will be readily apparent to those skilled in the art. Such compositions and methods for their preparation may be found, for example, in Remington's Pharmaceutical Sciences, 19th Edition (Mack Publishing Company, 1995).

A compound of formula (I) may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

A compound of formula (I) may also be used in fast-dissolving, fast-disintegrating dosage forms such as those described in Expert Opinion in Therapeutic Patents, 11 (6), 981-986, by Liang and Chen (2001).

For tablet dosage forms, depending on dose, a compound of formula (I) may make up from 1 weight % to 80 weight % of the dosage form, more typically from 5 weight % to 60 weight % of the dosage form. In addition to the compound of formula (I), tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% of a compound of formula (I), from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

A compound of formula (I) for use in a film may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, a compound of formula (I) may be used in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients in such a film include anti-oxidants, colorants, flavourings, flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed, sustained, pulsed, controlled, targeted and programmed release formulations.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

A compound of formula (I) may also be administered directly into the blood stream, into muscle, or into an internal organ. Suitable routes for such parenteral administration include intravenous, intraarterial, intraperitoneal, intrathecal, intraventricular, intraurethral, intrasternal, intracranial, intramuscular and subcutaneous delivery. Suitable means for parenteral administration include needle (including microneedle) injectors, needle-free injectors and infusion techniques.

Parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of a compound of formula (I) used in the preparation of a parenteral formulation may be increased by the use of appropriate formulation techniques, such as the incorporation of solubility-enhancing agents.

Formulations for parenteral administration may be formulated to be immediate and/or modified release. Modified release formulations include delayed, sustained, pulsed, controlled, targeted and programmed release formulations. Thus, a compound of formula (I) may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

A compound of formula (I) may also be administered topically to the skin or mucosa, i.e. dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm. Sci., 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed, sustained, pulsed, controlled, targeted and programmed release formulations.

A compound of the formula (I) can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of a compound of formula (I) comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of a compound of formula (I), a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of a compound of formula (I) per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted and programmed release formulations.

In the case of dry powder inhalers and aerosols, the dosage unit is determined by means of a valve which delivers a metered amount. Units in accordance with the invention are typically arranged to administer a metered dose or “puff”. The overall daily dose will be administered in a single dose or, more usually, as divided doses throughout the day.

A compound of formula (I) may be administered rectally or vaginally, e.g. in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed, sustained, pulsed, controlled, targeted and programmed release formulations.

A compound of formula (I) may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed, sustained, pulsed, controlled, targeted, or programmed release formulations.

A compound of formula (I) may be combined with a soluble macromolecular entitiy, such as a cyclodextrin or a suitable derivative thereof or a polyethylene glycol-containing polymer, in order to improve its solubility, dissolution rate, taste-masking, bioavailability and/or stability in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO-A-91/11172, WO-A-94/02518 and WO-A-98/55148.

For administration to human patients, the total daily dose of a compound of formula (I) is typically in the range of from 1 mg to 1000 mg depending, of course, on the mode of administration and the potency of the selected compound. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The ability of compounds of formula (I) to antagonise glutamate-evoked increases in calcium internalisation in an mGluR1 expressing cell line may be measured using the assay described below.

Chinese Hamster Ovary cells stably-transfected with the rat mGluR1a receptor (CHO-1a) were used in the assay. CHO-1a cells were seeded onto shallow 96 well plates and assayed at a confluency of 80-90%. Cells were loaded with Fluo-3-AM dye (8.8 μM final concentration; excitation 488 nm, emission 530 nm) in the presence of probenecid (2.5 mM), for 45-90 minutes at 37° C., after which time excess dye was removed by washing. The cell plate was then loaded onto a fluorometric imaging plate reader (FLIPr, Molecular Devices). Baseline readings were measured for 30 seconds. Test compounds were added by the FLIPr and allowed to equilibrate for 5 minutes before the addition of a 40 μM glutamate challenge (this was the approximate ED₉₀ for glutamate against the receptor expressed in these cells). Readings were taken for a further 2 minutes. Measurements were made of the area of peak obtained in response to the glutamate addition and inhibition curves were constructed. Glutamate dose-response curves were included on each experimental day to allow IC₅₀ values to be converted to modified Kᵢ values.

All the Examples described below were tested in this mGluR1 assay and were found to have an IC₅₀ of 10 μM or less. For instance, Examples 1 and 3 had binding affinities of 3 and 2 nM respectively.

An mGluR1 antagonist may be usefully combined with another pharmacologically active compound, or with two or more other pharmacologically active compounds, particularly in the treatment of pain. For example, an mGluR1 antagonist, particularly a compound of formula (I), or a pharmaceutically acceptable salt or solvate thereof, as defined above, may be administered simultaneously, sequentially or separately in combination with one or more agents selected from:
(i) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
(ii) a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac;
(iii) a barbiturate sedative, e.g. amobarbital, aprobarbital, butabarbital, butabital, mephobarbital, metharbital, methohexital, pentobarbital, phenobartital, secobarbital, talbutal, theamylal or thiopental;
(iv) a benzodiazepine having a sedative action, e.g. chlordiazepoxide, clorazepate, diazepam, flurazepam, lorazepam, oxazepam, temazepam or triazolam;
(v) an H₁ antagonist having a sedative action, e.g. diphenhydramine, pyrilamine, promethazine, chlorpheniramine or chlorcyclizine;
(vi) a sedative such as glutethimide, meprobamate, methaqualone or dichloralphenazone;
(vii) a skeletal muscle relaxant, e.g. baclofen, carisoprodol, chlorzoxazone, cyclobenzaprine, methocarbamol or orphrenadine;
(viii) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinone or cis-4-(phosphonomethyl)-2-piperidinecarboxylic acid;
(ix) an alpha-adrenergic, e.g. doxazosin, tamsulosin, clonidine or 4-amino-6,7-dimethoxy-2-(5-methanesulfonamido-1,2,3,4-tetrahydroisoquinol-2-yl)-5-(2-pyridyl) quinazoline;
(x) a tricyclic antidepressant, e.g. desipramine, imipramine, amytriptiline or nortriptiline;
(xi) an anticonvulsant, e.g. carbamazepine or valproate;
(xii) a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant or 3-[[2-methoxy-5-(trifluoromethoxy)phenyl]methylamino]-2-phenyl-piperidine (2S,3S);
(xiii) a muscarinic antagonist, e.g oxybutin, tolterodine, propiverine, tropsium chloride or darifenacin;
(xiv) a selective COX-2 inhibitor, e.g. celecoxib, rofecoxib or valdecoxib;
(xv) a non-selective COX inhibitor (preferably with GI protection), e.g. nitroflurbiprofen (HCT-1026);
(xvi) a coal-tar analgesic, in particular paracetamol;
(xvii) a neuroleptic such as droperidol;
(xviii) a vanilloid receptor agonist (e.g. resinferatoxin) or antagonist (e.g. capsazepine);
(xix) a beta-adrenergic such as propranolol;
(xx) a local anaesthetic such as mexiletine;
(xxi) a corticosteriod such as dexamethasone
(xxii) a 5-HT receptor agonist or antagonist, particularly a 5-HT_{1B/1D} agonist such as eletriptan, sumatriptan, naratriptan, zolmitriptan or rizatriptan;
(xxiii) a cholinergic (nicotinic) analgesic;
   (xxiv) Tramadol (trade mark);
(xxv) a PDEV inhibitor, such as sildenafil, vardenafil, taladafil, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}4-ethylpiperazine, or N-[1-(2-ethoxyethyl)-5-(N-ethyl-N-methylamino)-7-(4-methylpyridin-2-ylamino)-1H-pyrazolo[4,3-d]pyrimidine-3-carbonyl]methanesulfonamide;
(xxvi) an alpha-2-delta ligand such as gabapentin, pregabalin, (1α,3α, 5α)(3-amino-methyl-bicyclo[3.2.0]hept-3-yl)-acetic acid, (3S,5R)-3-Aminomethyl-5-methyl-heptanoic acid, (3S,5R)-3-amino-5-methyl-heptanoic acid, (3S, 5R)-3-Amino-5-methyl-octanoic acid, (2S,4S)-4-(3-chlorophenoxy)proline or (2S,4S)-4-(3-fluorobenzyl)proline;
(xxvii) a canabinoid;
(xxviii) metabotropic glutamate subtype 1 receptor (mGluR1) antagonist;
(xxix) a serotonin reuptake inhibitor such as sertraline;
(xxx) a noradrenaline reuptake inhibitor, especially a selective noradrenaline reuptake inhibitor such as (S,S)-reboxetine;
(xxxi) an inducible nitric oxide synthase (iNOS) inhibitor such as S-[2-[(1-iminoethyl)amino]ethyl]-2-methyl-L-cysteine or (2S,5Z)-2-amino-2-methyl-7-[(1-iminoethyl)amino]-5-heptenoic acid;
(xxxii) an acetylcholine esterase inhibitor such as donepezil;
(xxxiii) a dopamine type 2 (D2) antagonist such as ziprazidone;
(xxxiv) an prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)carbonyl]-4-methylbenzenesulfonamide or 4-[(1S)-1-({[5-chloro-2-(3-fluorophenoxy)pyridin-3-yl]carbonyl}amino)ethyl]benzoic acid;
   and the pharmaceutically acceptable salts and solvates thereof.

Where a combination of active compounds is to be administered, two or more pharmaceutical compositions may conveniently be combined in the form of a kit suitable for coadministration of the compositions.

Such a kit comprises two or more separate pharmaceutical compositions, at least one of which contains an mGluR1 antagonist, particularly a compound of formula (I), and means for separately retaining said compositions, such as a container, divided bottle, or divided foil packet. An example of such a kit is the familiar blister pack used for the packaging of tablets, capsules and the like.

Such a kit is particularly suitable for administering different dosage forms, for example, oral and parenteral formulations, for administering separate compositions at different dosage intervals, or for titrating separate compositions against one another. To assist compliance, the kit typically comprises directions for administration and may be provided with a so-called memory aid.

It will be appreciated that what the invention provides, and what will be claimed, includes:
(i) a compound of formula (I) or a pharmaceutically acceptable salt or solvate thereof;
(ii) a process for the preparation of a compound of formula (I) or a pharmaceutically acceptable salt or solvate thereof;
(iii) a pharmaceutical composition including a compound of formula (I) or a pharmaceutically acceptable salt or solvate thereof, together with a pharmaceutically acceptable excipient;
(iv) a compound of formula (I) or a pharmaceutically acceptable salt, solvate or composition thereof, for use as a medicament;
(v) the use of a compound of formula (I) or of a pharmaceutically acceptable salt, solvate or composition thereof, for the manufacture of a medicament to treat a disease for which an mGluR1 receptor antagonist is indicated;
(vi) the use of a compound of formula (I) or of a pharmaceutically acceptable salt, solvate or composition thereof, for the manufacture of a medicament for the treatment of pain, particularly nociceptive pain, or a neurodegenerative disease;
(vii) a method of treatment of a mammal, including a human being, with an mGluR1 antagonist including treating said mammal with an effective amount of a compound of formula (I) or with a pharmaceutically acceptable salt, solvate or composition thereof;
(viii) a method of treatment of a mammal, including a human being, to treat pain or a neurodegenerative disease, including treating said mammal with an effective amount of a compound of formula (I) or with a pharmaceutically acceptable salt, solvate or composition thereof;
(ix) certain novel intermediates disclosed herein; and
(x) a combination of a compound of formula (I) and one or more further pharmacologically active compounds.

The following Examples illustrate the preparation of compounds of formula (I).

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ESI) or atmospheric pressure chemical ionisation (APCl). The following abbreviations have the indicated meaning: CDCl₃, deuterochloroform; D₆-DMSO, deuterodimethylsulphoxide; CD₃OD, deuteromethanol; THF, tetrahydrofuran. ‘Ammonia’ refers to a concentrated solution of ammonia in water possessing a specific gravity of 0.88. Where thin layer chromatography (TLC) has been used it refers to silica gel TLC using silica gel 60 F₂₅₄ plates, R_{f} is the distance travelled by a compound divided by the distance travelled by the solvent front on a TLC plate. Microwave radiation was performed using machines with a power range of 15 to 300 W at 2.45 GHz, the actual power supplied varying during the course of the reaction to maintain a constant temperature.

### EXAMPLE 1

1-(4-Cycloheptylamino-pyrido[3,4-d]pyrimidin-6-yl)-piperidin-4-ol

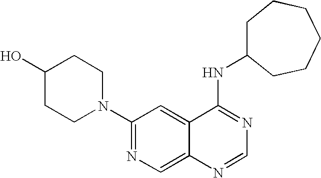

The fluoro compound of Preparation 1 (50 mg, 0.192 mmol) was treated with piperidin-4-ol (553 mg, 4.8 mmol) and the reaction mixture was stirred at 100° C. for 3 hours and then at 130° C. for 6 hours. The cooled reaction mixture was partitioned between dichloromethane (25 ml) and water (25 ml), the layers were separated and the organic phase was filtered through a hydrophobic frit. The organic solution was concentrated in vacuo and the crude product was dissolved in dichloromethane and purified by column chromatography on silica gel eluting with dichloromethane:methanol:ammonia 100:0:0 to 97:3:0.7 (by volume) to yield the title product as a yellow oil, 4.2 mg.

¹H NMR(CD₃OD, 400 MHz): 1.52-1.81(m, 12H), 1.93-1.99(m, 2H), 2.02-2.10(m, 2H), 3.18-3.25(m, 2H), 3.83-3.90(m, 1H), 4.19(m, 2H), 4.37(m, 1H), 7.25(m, 1H), 8.22(m, 1H), 8.67(m, 1H).

MS ((ESI+)) m/z 342 [MH]⁺.

### EXAMPLE 2

N*4*-Cycloheptyl-N*6*-(3-morpholin-4-yl-propyl)-pyrid[3,4-d]pyrimidine-4,6-diamine

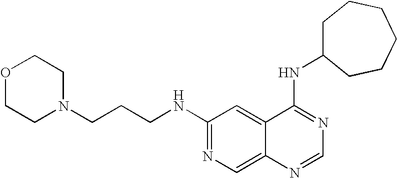

The title compound was prepared by a method similar to that described for Example 1 using 3-morpholin-4-yl-propylamine and the fluoro compound of Preparation 1 as starting materials. The title compound was isolated as a yellow oil.

¹H NMR(CD₃OD, 400 MHz): 1.57-1.80(m, 10H), 1.86(m, 2H), 2.08(m, 2H), 2.49(m, 6H), 3.36(m, 2H), 3.89(m, 4H), 4.37(m, 1H), 6.91(m, 1H), 8.19(m, 1H), 8.56(m, 1H).

MS ((ESI+)) m/z 385 [MH]⁺.

### EXAMPLE 3

3-(4-Cycloheptyl-pyrido[3,4-d]pyrimidin-6-ylamino-propan-1-ol

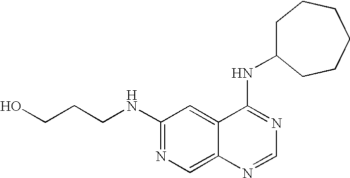

The title compound was prepared by a method similar to that described for Example 1 using 3-amino-propan-1-ol and the fluoro compound of Preparation 1 as starting materials.

¹H NMR(CD₃OD, 400 MHz):1.50-1.78(m, 10H), 1.87(m, 2H), 2.02(m, 2H), 3.20(m, 2H), 3.69(m, 2H), 4.33(m, 1H), 4.88(m, 1H), 6.97(m, 1H) 8.16(m, 1H), 8.55(m, 1H).

MS (ESI+) m/z 316 [MH]⁺.

### EXAMPLE 4

N*4*-Cyclohexyl-N*6*-(2-morpholin-4-yl-ethyl)pyrido[3,4-d]pyrimidine-4,6-diamine

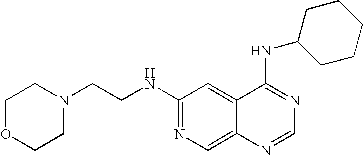

The fluoro compound of Preparation 2 (150 mg, 0.61 mmol) was treated with N-(2-aminoethyl)-morpholine (2 ml, 15.2 mmol) and the solution was heated at 110° C. for 7 hours. The cooled reaction mixture was partitioned between dichloromethane (25 ml) and water (25 ml) and the phases were separated. The organic phase was dried by filtration through a hydrophobic frit and then concentrated in vacuo. The crude product was purified by column chromatography on silica gel eluting with dichloromethane:methanol:ammonia 100:0:0 to 94:6:0.6 (by volume) to yield the title compound (29.9 mg) as a bright yellow oil.

¹H NMR(CD₃OD, 400 MHz): 1.28(m, 1H), 1.38-1.52(m, 4H), 1.74(m, 1H), 1.85(m, 2H), 2.04(m, 2H), 2.55(m, 4H), 2.67(t, 2H), 3.45(t, 2H), 3.72(t, 4H), 4.17(m, 1H), 6.94(m, 1H), 8.20m, 1H), 8.60(s, 1H).

MS (ESI+) m/z 379 [MNa]⁺.

### EXAMPLE 5

N*4*-Cyclohexyl-N*6*-(3-imidazol-1-yl-propyl)-pyrido[3,4-d]pyrimidine-4,6-diamine

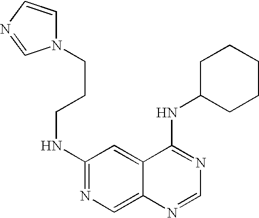

The title compound was prepared by a method similar to that described for Example 4 using N-(3-aminopropyl)imidazole and the fluoro compound of Preparation 2 as starting materials.

¹H NMR(CD₃OD, 400 MHz): 1.24(m, 1H), 1.38(m, 4H), 1.71(m, 1H), 1.86(m, 2H), 2.06(m, 2H), 2.11(m, 2H), 3.33(m, 2H), 4.13(m, 3H), 6.86(m, 1H), 6.97(m, ₁H), 7.12(m, 1H), 7.66(m, 1H), 8.19(m, 1H), 8.60(m, 1H).

MS (ESI+) m/z 352 [MH]⁺.

### EXAMPLE 6

Cyclohexyl-[6-(tetrahyrdo-furan-3-ylmethoxy)-Pyrido[3,4-d]pyrimidin-4-yl]-amine

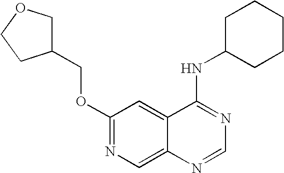

The title compound was prepared by a method similar to that described for Example 4 using (tetrahydro-furan-3-yl)-methanol and the fluoro compound of Preparation 2 as starting materials.

¹H NMR(CD₃OD, 400 MHz): 1.23(m, 1H), 1.42-1.57(m, 4H), 1.71-1.90(m, 4H), 2.08(m, 2H), 2.25(m, 1H), 2.88(m, 1H), 3.72(m, 2H), 3.94(m, 2H), 4.23(m, 2H), 4.43(m, 1H), 7.54(m, 1H), 8.48(m, 1H), 8.76(s, 1H).

MS ESI-m/z 327 [M-H]⁻.

### EXAMPLE 7

(6S)-5-(4-Cyclohexylamino-pyrido[3,4-d]pyrimidin-6-yloxymethyl)-pyrrolidin-2-one

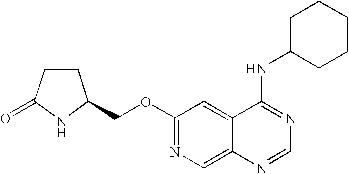

The title compound was prepared by a method similar to that described for Example 4 using (S)-5-(hydroxymethyl)-2-pyrrolidone and the fluoro compound of Preparation 2 as starting materials.

¹H NMR(CD₃OD, 400 MHz): 1.28(m, 1H), 1.44(m, 4H), 1.73(m, 1H), 1.86(m, 2H), 2.07(m, 3H), 2.38(m, 2H), 2.46(m, 1H), 4.10(m, 1H), 4.21(m, 1H), 4.36(m, 1H), 4.45(m, 1H), 7.52(m, 1H), 8.36(m, 1H), 8.72(m, 1H).

MS (ESI+) m/z 342 [MH]⁺.

### EXAMPLE 8

4-(4-Cyclohexylamino-pyrido[3,4-d]pyrimidin-6-yloxy)-cyclohexanol

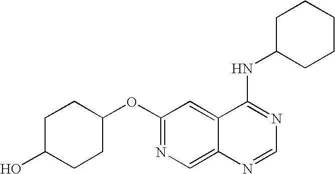

The title compound was prepared by a method similar to that described for Example 4 using cyclohexane-1,4-diol as the reagent.

¹H NMR(CD₃OD, 400 MHz): 1.24(m, 1H), 1.37-1.62(m, 7H), 1.76(m, 3H), 1.83(m, 2H), 2.00(m, 4H), 2.16(m, 1H), 3.71(m, 1H), 4.17(m, 1H), 5.07(m, 1H), 7.42(m, 1H), 8.36(m, 1H), 8.72(m, 1H).

MS (ESI+) m/z 343 [MH]⁺.

### EXAMPLE 9

N*4*-Cyclohexyl-N*6*-((2S) tetrahydro-furan-2-ylmethyl)-pyrido[3,4-d]pyrimidine-4,6-diamine

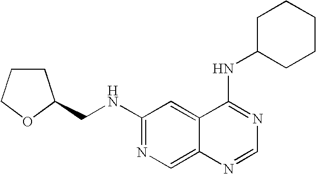

The title compound was prepared by a method similar to that described for Example 4 using (S)-tetrahydrofurfurylamine and the fluoro compound of Preparation 2 as starting materials. The title product was isolated as an orange oil.

MS (ESI+) m/z 350 [MNa]⁺.

### EXAMPLE 10

N-[2-(4-Cyclohexylamino-pyrido[3,4-d]pyrimidin-6-ylamino)-ethyl]-methanesulphonamide

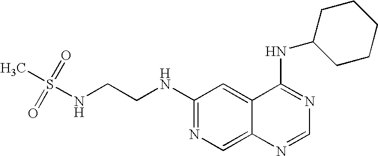

The amine of Preparation 5 (50 mg, 0.175 mmol) was added to a solution of triethylamine (29 μL, 0.21 mmol) in dichloromethane (0.5 ml), at 0° C., under an atmosphere of nitrogen. To this mixture was added a solution of methanesulphonyl chloride (14 μL, 0.175 mmol) in dichloromethane (0.5 ml). The reaction mixture was left at 0° C. for 10 minutes, stirred at room temperature for 40 minutes and then concentrated in vacuo. The crude product was purified by column chromatography on silica gel eluting with dichloromethane:methanol:ammonia 100:0:0 to 90:10:1 (by volume) to yield the title product as an oil (28.7 mg, 45% yield).

¹H NMR(CD₃OD, 400 MHz): 1.31(m, 8H), 1.72(m, 1H), 1.84(m, 2H), 2.06(m, 2H), 2.92(m, 2H), 3.48(m, 2H), 4.14(m, 1H), 6.99(m, 1H), 8.22(m, 1H), 8.60(m, 1H).

MS (ESI+) m/z 365 [MH]⁺.

### EXAMPLE 11

N*4*-(4,4-Difluoro-cyclohexyl)-N*6*-(2-morpholin-4-yl-ethyl)-pyrido[3,4-d]pyrimidine-4,6-diamine

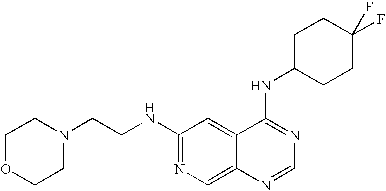

The fluoro compound of Preparation 3 (50 mg, 0.18 mmol) was treated with N-(2-aminoethyl)-morpholine (0.5 ml, 3.8 mmol) and the reaction mixture was heated to 130° C. for 2 hours. Water (30 ml) and dichloromethane (20 ml) were added to the reaction mixture and the organic layer was separated. The organic layer was concentrated in vacuo and the crude product was purified by column chromatography on silica gel eluting with ethyl acetate to yield the title product (42.2 mg).

¹H NMR(CD₃OD, 400 MHz): 1.78(m, 2H), 2.05(m, 5H), 2.69(m, 5H), 2.75(m, 2H), 3.52(m, 2H), 3.71(m, 4H), 4.44(m, 1H), 6.97(m, 1H), 8.23(m, 1H), 8.66(m, 1H).

MS (ESI+) m/z 393 [MH]⁺.

### EXAMPLE 12

N*4*-(4,4-Difluoro-cyclohexyl)-N*6*-((2S)-tetrahydro-furan-2-ylmethyk)-pyrido[3,4-d]pyrimidine-4,6-diamine

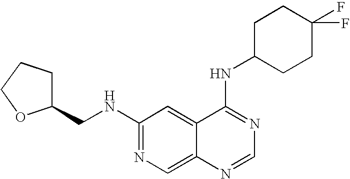

The title compound was prepared by a method similar to that described for Example 11 using (S)-tetrahydrofurfurylamine and the fluoro compound of Preparation 3 as starting materials.

¹H NMR(CD₃OD, 400 MHz): 1.64-2.09(brm, 12H), 3.36(m, 1H), 3.45(m, 1H), 3.77(dd, 1H), 3.88(dd, 1H), 4.12(m, 1H), 4.29(m, 1H), 6.91(m, 1H), 8.22m, 1H), 8.59(m, 1H).

MS (ESI+) m/z 364 [MH]⁺.

### EXAMPLE 13

N*4*-Indan-2-1-N*6*-(2-methoxy-ethyl)-pyrido[3,4-d]pyrimidine-4,6-diamine

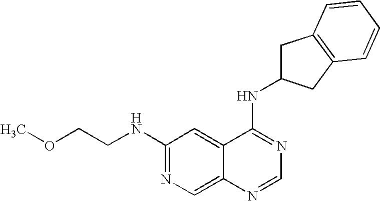

The fluoro compound of Preparation 4 (280 mg, 0.99 mmol) and 2-methoxy-ethylamine (750 mg, 9.98 mmol) were dissolved in dimethyl sulphoxide (18 ml) and the reaction mixture was stirred at 80° C. for 18 hours and then at 100° C. for 48 hours. The reaction mixture was concentrated in vacuo and the crude product was purified by column chromatography on silica gel eluting with dichloromethane:methanol 95:5 (by volume) to yield the title product as a brown solid (217 mg, 64.8% yield).

¹H NMR(DMSO-d₆, 400 MHz): 3.03(dd, 2H), 3.28(s, 3H), 3.38(m, 4H), 3.50(m, 2H), 5.00(m, 1H), 6.40(m, 1H), 6.96(s, 1H), 7.15(m, 2H), 7.25(m, 2H), 8.14(d, 1H), 8.27(s, 1H), 8.60(s, 1H).

MS (APCl+) m/z 336 [MH]⁺.

### EXAMPLE 14

2-{2-[4-(Indan-2-ylamino)-pyrido[3,4-d]pyrimidin-6-ylamino]-ethoxy}-ethanol

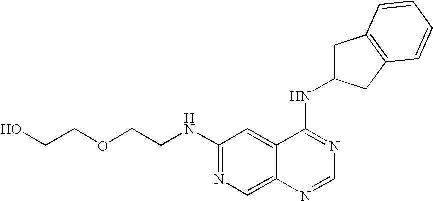

The title compound was prepared by a method similar to that described for Example 13 using 2-(2-amino-ethoxy)-ethanol as the relevant starting material.

¹H NMR(DMSO-d₆, 400 MHz): 3.03(m, 2H), 3.32-3.53(m, 8H), 4.59(m, 2H), 4.61(m, 1H), 4.96(m, 1H), 6.46(m, 1H), 6.94(m, 1H), 7.14(m, 2H), 7.24(m, 2H), 8.16(m, 1H), 8.27(m, 1H), 8.61 (m, 1H).

MS (APCl+) m/z 366 [MH]⁺.

### EXAMPLE 15

(6-Azetidin-1-yl-pyrido[3,4-d]pyrimidin-4-yl)-indan-2-yl-amine

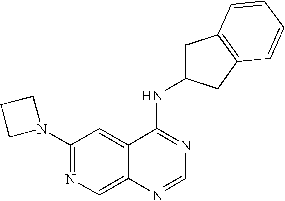

The title compound was prepared by a method similar to that described for Example 13 using azetidine and the fluoro compound of Preparation 4 as starting materials. The title compound was isolated as a yellow solid.

¹H NMR(DMSO-D₆, 400 MHz): 2.32(m, 2H), 3.03(m, 2H), 3.37(m, 2H), 3.96(m, 5H), 5.96(m, 1H), 6.92(m, 1H), 7.14(m, 1H), 7.23(m, 1H), 8.21(m, 1H), 8.31 (m, 1H), 8.66(m, 1H).

MS (APCl+) m/z 318 [MH]⁺.

### EXAMPLE 16

N*6*-Indan-2-yl-N*4*-(tetrahydro-furan-2-ylmethyl)-pyrido[3,4-d]pyrimidine-6-diamine

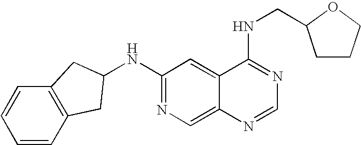

The fluoro compound of Preparation 6 (0.15g, 0.60 mmol) was added to a solution of 2-aminoindane (0.51g, 3.0 mmol) in dimethylsulfoxide (2 ml) and the resulting mixture was heated to 220° C. for 1500 seconds using microwave irradiation. The mixture was diluted with water (25 ml) and extracted with ethyl acetate (3×25 ml). The combined organic layers were dried (MgSO₄) and concentrated in vacuo. The crude product was purified by column chromatography on silica gel, eluting with ethyl acetate, to yield the title product as a brown solid (44 mg, 20%).

¹HNMR(CD₃OD): 1.69(m, 1H), 1.95(m, 2H), 2.06(m, 1H), 2.90(dd, 2H), 3.44(dd, 2H), 3.61(dd, 1H), 3.77(m, 2H), 3.91(m, 2H), 4.25(m, 1H), 4.58(m, 1H), 6.91(s, 1H), 7.14(m, 2H), 7.23(m, 2H), 8.22(s, 1H), 8.64(s, 1H).

MS (APCl+) m/z 362 [MH]⁺.

Microanalysis: Found C, 69.62; H, 6.41; N, 19.43. C₂₁H₂₃N₅O requires C, 69.78; H, 6.41; N, 19.38%.

### EXAMPLE 17

N*6*-Cyclohexyl-N*4*-(tetrahydro-furan-2-ylmethyl)-pyrido[3,4-d]pyrimidine-4,6-diamine

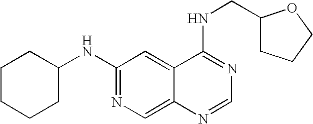

This compound was prepared by a method similar to that described for Example 16 using cyclohexylamine as the appropriate starting material. The title compound was prepared as a yellow solid (21%).

¹HNMR(CD₃OD): 1.20-1.60(m, 2H), 1.70(m, 2H), 1.82(m, 2H), 1.90-2.14(m, 6H), 3.55-3.66(m, 3H), 3.76(m, 2H), 3.91(dd, 1H), 4.25(m, 1H), 6.81(s, 1H), 8.18(s, 1H), 8.58(s, 1H).

MS (APCl+) m/z 328 [MH]⁺.

Microanalysis: Found C, 65.88; H, 7.64; N, 21.45. C₁₈H₂₅N₅O requires C, 66.03; H, 7.70; N, 21.39%.

### EXAMPLE 18

N*6*-(2-Morpholin-4-yl-ethyl)-N*4*-(tetrahydro-furan-2-ylmethyl)-pyrido[3,4-d]pyrimidine-4,6-diamine 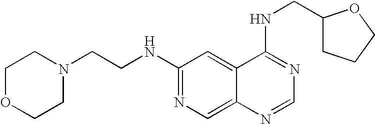
This compound was prepared by a method similar to that described for Example 16 using 2-(4-morpholino)-ethylamine as the appropriate starting material. The title compound was prepared as a yellow solid (28%).

¹HNMR(CD₃OD): 1.69(m, 1H), 1.95(m, 2H), 2.06(m, 1H), 2.55(m, 4H), 2.68(t, 2H), 3.46(t, 2H), 3.62(dd, 2H), 3.71-3.81(m, 6H), 3.91(dd, 1H), 4.24(m, 1H), 6.87(s, 1H), 8.22(s, 1H), 8.62(s, 1H).

MS (APCl+) m/z 359 [MH]⁺.

Microanalysis: Found C, 59.93; H, 7.31; N, 23.23. C₁₈H₂₆N₆O₂ requires C, 60.32; H, 7.31; N, 23.45%.

### EXAMPLE 19

1-[4-(4-Cyclohexylamino-pyrido[3,4-d]pyrimidin-6-yl)-piperazinyl]-ethanone hydrochloride

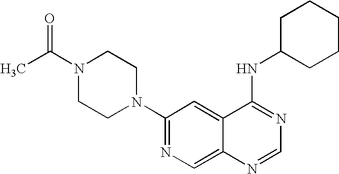

The fluoro compound of Preparation 2 (200 mg, 0.81 mmol) was dissolved in N-methylpyrrolidinone (5 ml) and the resulting solution was treated with N-ethyldiisopropylamine (0.2 ml, 1.22 mmol) and N-acetylpiperazine (230 mg, 1.78 mmol) and heated at 120° C. for 5 days. The reaction was allowed to cool and poured into water (75 ml). The aqueous mixture was extracted with ethylacetate (2×50 ml) and the combined organic extracts were dried (Na₂SO₄) and concentrated in vacuo. The crude product was purified by column chromatography on silica gel eluting with dichloromethane:methanol 100:0 to 90:10 (by volume) to afford the product as an oil. The product was dissolved in hot ethanol (2 ml) and a solution of hydrogen chloride in diethylether (1M) was added until a precipitate appeared. The yellow solid was collected by filtration to afford the product as the hydrochloride salt (77 mg, 24% yield).

¹HNMR(CD₃OD, 400 MHz): 1.25(m, 1H), 1.38-1.62(br m, 4H), 1.73(m, 1H), 1.84(m, 2H), 2.03(m, 2H), 2.18(s, 3H), 3.60-3.80(m, 6H), 3.85(m, 2H), 4.48(m, 1H), 7.53(br s, 1H), 8.51 (s, 1H), 8.77(s, 1H).

MS (APCl+) m/z 343 [MH]⁺.

### EXAMPLE 20

1-[2-(4-Cyclohexylamino-pyrido[3,4-d]pyrimidin-6-yloxy)-ethyl]-pyrrolidin-2 one hydrochloride hemihydrate

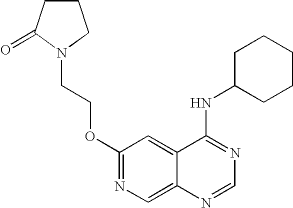

A solution of the fluoro compound of Preparation 2 (400 mg, 1.6 mmol) in 1-(2-hydroxy-ethyl)-pyrrolidin-2-one (3 ml) was treated with potassium tert-butoxide (600 mg, 8.1 mmol) and heated at 100° C. for 16 hours. The reaction was allowed to cool and then poured into water (500 ml). The aqueous mixture was extracted with ethylacetate (2×200 ml) and the combined organic extracts were washed with brine (2×100 ml), dried (Na₂SO₄) and concentrated in vacuo. The crude product was purified by column chromatography on silica gel eluting with dichloromethane:methanol 100:0 to 90:10 (by volume) to afford the product as an oil. The product was dissolved in hot ethanol (10 ml) and a solution of hydrogen chloride in diethylether (2 ml of a 1M solution, 2 mmol) was added. The resulting solution was evaporated and a solid was obtained following trituration with diethylether. The pale solid was collected by filtration to afford the product as the hydrochloride salt hemihydrate (375 mg, 58%).

¹HNMR(d₆-DMSO, 400 MHz): 1.18(q, 1H), 1.35(q, 2H), 1.50(q, 2H), 1.65(d, 1H), 1.79(d, 2H), 1.95(m, 4H), 2.18(t, 2H), 3.41(t, 2H), 3.59(t, 2H), 4.30(m, 1H), 4.42(t, 2H), 8.00(s, 1H), 8.79(s, 1H), 8.88(s, 1H), 9.95(d, 1H).

MS (APCl+) m/z 356 [MH]⁺.

Microanalysis: Found C, 56.99; H, 6.82; N, 17.56. C₁₉H₂₅N₅O₂.HCl.0.5H₂O requires C, 56.92; H, 6.79; N, 17.47%.

### EXAMPLE 21

[6-(4-Methoxy-piperidin-1-yl)-pyrido[3,4-d]pyrimidin-4-yl]-(6-methyl-pyridin-2-ylmethyl)-amine

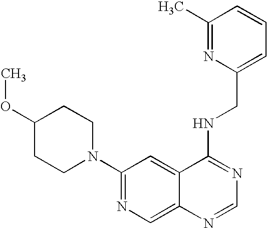

The fluoro compound of Preparation 8 (0.2g, 0.74 mmol) was added to a mixture of 4-methoxypiperidine (0.5 ml, 4 mmol), N,N-diisopropylethylamine (0.5 ml, 2.8 mmol) and N-methylpyrrolidinone (1.5 ml) and the reaction was heated to 90° C. for 60 hours. The mixture was allowed to cool and poured into water (35 ml). The resulting yellow solid was collected by filtration and dried in vacuo to yield the title product (175 mg, 65%).

¹HNMR(CDCl₃, 400 MHz): 1.63(m, 2H), 1.93(m, 2H), 2.60(s, 3H), 3.11(m, 2H), 3.38(s, 3H), 3.42(m, 1H), 3.82(m, 2H), 4.87(d, 2H), 6.57(s, 1H), 7.12(d, 1H), 7.27(d, 1H), 7.64(t, 1H), 8.10(br s, 1H), 8.45(s, 1H), 8.83(s, 1H).

MS (APCl+) m/z 366 [MH]⁺.

### EXAMPLE 22

[6-(4-Methoxy-piperidin-1-yl)-pyrido[3,4-d]pyrimidin-4-yl]-pyridin-2-ylmethyl-amine

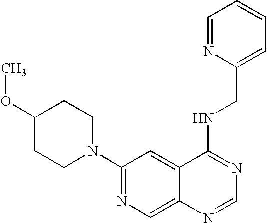

The fluoro compound of Preparation 7 (0.2g, 0.74 mmol) was added to a mixture of 4-methoxypiperidine (0.5 ml, 4 mmol), N,N-diisopropylethylamine (0.5 ml, 2.8 mmol) and N-methylpyrrolidinone (1.5 ml) and the reaction was heated to 90° C. for 60 hours. The mixture was allowed to cool and poured into water (35 ml). The resulting yellow solid was collected by filtration and dried in vacuo to yield the title product (150 mg, 58%).

¹HNMR(CDCl₃): 1.63(m, 2H), 1.94(m, 2H), 1.82(m, 2H), 3.13(m, 2H), 3.39(s, 3H), 3.41(m, 1H), 3.86(dt, 2H), 4.92(d, 2H), 6.60(s, 1H), 7.27(d, 1H), 7.44(d, 1H), 7.74(t, 1H), 7.98(br s, 1H), 8.46(s, 1H), 8.61(d, 1H), 8.86(s, 1H).

MS (APCl+) m/z 351 [MH]⁺.

### EXAMPLE 23

[6-(4,4-Difluoro-piperidin-1-yl)-pyrido[3,4-d]pyrimidin-4-yl]-pyridin-2-ylmethyl-amine

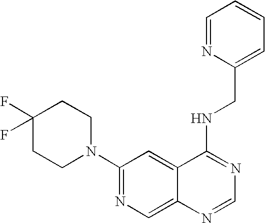

The fluoro compound of Preparation 7 (0.2g, 0.74 mmol) was added to a mixture of 4,4-difluoropiperidine hydrochloride (0.4g, 2.5 mmol), N,N-diisopropylethylamine (1 ml, 5.6 mmol), tetraethylammonium fluoride hydrate (0.2g, 1. mmol) and dimethylsulfoxide (5 ml) and the reaction was heated to 130° C. for 60 hours. The mixture was allowed to cool and poured into water (50 ml) and the aqueous phase was extracted with ethyl acetate (2×50 ml). The combined organic phases were dried (Na₂SO₄) and evaporated. The resulting residue was purified by flash chromatography to afford an oil. This oil was dissolved in N-methylpyrrolidinone (0.5 ml) and added dropwise with stirring to water (50 ml). The resulting solid was collected by filtration and dried in vacuo to yield the title product (95 mg, 65%).

¹HNMR(CDCl₃, 400 MHz): 2.01(m, 4H), 3.62(t, 4H), 4.93(d, 2H), 6.67(s, 1H), 7.30(t, 1H), 7.47(d, 1H), 7.78(t, 1H), 8.16(br s, 1H), 8.48(s, 1H), 8.63(d, 1H), 8.86(s, 1H).

MS (APCl+) m/z 357 [MH]⁺.

### EXAMPLE 24

N*4*-Cycloheptyl-N*6*-(3-dimethylamino-propyl)-pyrido[3,4-d]pyrimidine-4,6-diamine

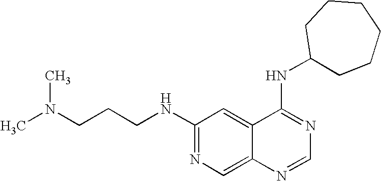

The fluoro compound of Preparation 1 (0.5 g, 1.9 mmol) was added to a solution of N,N-dimethylaminopropylamine (0.75 ml, 5.96 mmol) and triethylamine (0.5 ml, 3.8 mmol) in M-methylpyrrolidinone (1.55 ml) in a microwave reaction vial. The mixture was heated under microwave irradiation to 180° C. for 800 seconds. The mixture was allowed to cool and poured into water (30 ml) and the aqueous phase was extracted with ethyl acetate (3×50 ml). The combined organic phases were washed with brine (50 ml), dried (Na₂SO₄) and evaporated. The resulting residue was purified by flash chromatography (gradient elution, 98:2 to 90:10 by volume dichloromethane:10% ammonia in methanol) to afford a yellow oil which solidified on standing. This solid was triturated with ether and dried to yield the title product as a yellow solid (330 mg, 51%).

¹HNMR(CDCl₃, 400 MHz): 1.50-1.90(m, 10H), 2.00-2.20(m, 4H), 2.24(s, 6H), 2.51(t, 2H), 3.37(t, 2H), 4.48-4.52(m, 1H), 5.40(br s, 1H), 5.60(d, 1H), 6.22(s, 1H), 8.41 (s, 1H), 8.80(s, 1H).

MS (ES+) m/z 343 [MH]⁺.

### EXAMPLE 25

Cycloheptyl-[6-(4-dimethylaminomethyl-piperidin-1-yl)-pyrido[3,4-d]pyrimidin-4-yl]-amine

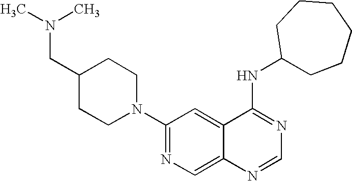

A mixture of the fluoro compound of Preparation 1 (400 mg, 1.54 mmol) and dimethyl-piperidin-4-ylmethyl-amine (655 mg, 4.61 mmol, see European Journal of Medicinal Chemistry, 2002, 37(6), 487-501) was microwaved at 200° C. for 30 minutes as a solution in 1-methyl-pyrrolidin-2-one (3 ml). The reaction mixture was poured in water (30 ml) and 510 mg of yellow solid precipitated. Chromatography on silica gel (eluent: gradient from 95/5 by volume dichloromethane/methanol to 90/10/1 by volume dichloromethane/methanol/ammonia) yielded the title compound as a yellow solid (480 mg, 82%).

¹H-NMR (400 MHz, CDCl₃): δ=1.29 (dq, 2H), 1.65 (m, 11H), 1.90 (d, 2H), 2.13 (m, 2H), 2.21 (d, 2H), 2.26 (s, 6H), 2.86 (t, 2H), 4.32 (d, 2H), 4.39 (m, 1H), 5.60 (d, 1H), 6.44 (s, 1H), 8.47 (s, 1H), 8.90 (s, 1H).

MS (ES+) m/z383 [MH]⁺.

### EXAMPLE 26

Cycloheptyl-[6-(3-dimethylamino-propoxy)-pyrido[3,4-d]pyrimidin-4-yl]-amine

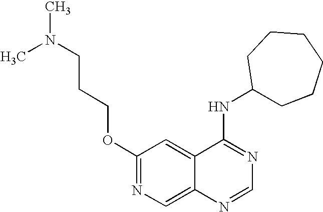

A mixture of the fluoro compound of Preparation 1 (100 mg, 0.39 mmol), 3-dimethylamino-propan-1-ol (158 mg, 1.54 mmol) and 1-methylpyrrolidin-2-one (3 ml) was microwaved at 200° C. for 10 minutes. The reaction mixture was partitioned between ethyl acetate and water and the organic phase was washed twice with water and concentrated under vacuum. Chromatography on silica gel (eluting with 95/4.5/0.5 by volume dichloromethane/methanol/ammonia) yielded the title compound as a white solid (50 mg, 39%).

¹H-NMR (400 MHz, CDCl₃): 1.5-1.8(m, 8H), 1.94(m, 2H), 2.05(m, 2H), 2.22(s, 6H), 2.43(t, 2H), 4.37(t, 2H), 5.49(d, 1H), 6.75(s, 1H), 8.49(s, 1H), 8.83(s, 1H).

LCMS (ES+) m/z 344.5 [MH]⁺.

### EXAMPLE 27

Cycloheptyl-[6-(2-morpholin-4-yl-ethoxy)-pyrido[3,4-d]pyrimidin-4-yl]-amine 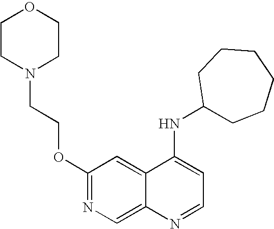
4-(2-Hydroxyethyl)-morpholine (680 mg, 5.2 mmol) was added to a solution of potassium tert-butoxide (534 mg, 4.76 mmol) in 1-methylpyrrolidin-2-one (3 ml). After stirring for 5 minutes at room temperature the fluoro compound of Preparation 1 (0.45g, 1.74 mmol) was added and the reaction was heated with microwave radiation at 180° C. for 400 seconds. The reaction mixture was poured into water (250 ml) and the resulting solid was collected by filtration to afford the title compound as a white solid (500 mg, 77%).

¹H-NMR (400 MHz, CDCl₃): 1.5-1.8(m, 10H), 2.1(m, 2H), 2.6(m, 4H), 2.8(t, 2H), 3.7(m, 4H), 4.34.4(m, 1H), 4.57(t, 2H), 5.6(d, 1H), 6.84(s, 1H), 8.57(s, 1H), 8.86(s, 1H).

LCMS (ES+): m/z 372 [MH]⁺.

### EXAMPLE 28

[6-(2-Aziridin-1-yl-ethoxy)-pyrido[3,4-d]pyrimidin-4-yl]-cycloheptyl-amine

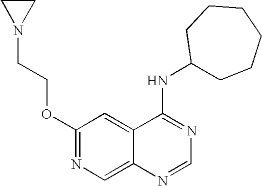

This compound was prepared by a method similar to that described for Example 27 using the fluoro compound of Preparation 1 and 1-(hydroxyethyl)aziridine as starting materials. The title product was obtained as a white solid in 50% yield.

¹H-NMR (400 MHz, CDCl₃): 1.2(m, 2H), 1.5-1.7(m, 10H), 2.1(m, 2H), 2.5-2.7(m, 4H), 4.4(m, 1H), 4.54(t, 2H), 5.6(d, 1H), 6.9(s, 1H), 8.58(s, 1H), 8.9(s, 1H).

LCMS (ES+): m/z 328 [MH]⁺.

### EXAMPLE 29

N*6*-(3-Dimethylamino-propyl)-N*4*-(trans-4-methyl-cyclohexyl)-pyrido[3,4-d]pyrimidine-4,6-diamine

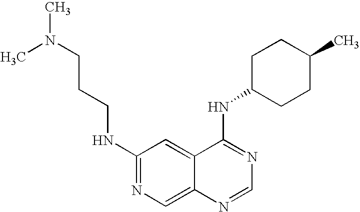

This compound was prepared by a method similar to that described for Example 4 using the fluoro compound of Preparation 9 and N,N-dimethylaminopropylamine as starting materials. The title product was obtained as a yellow solid in 60% yield.

¹H-NMR (400 MHz, CDCl₃): 0.91(d, 3H), 1.16(qd, 2H), 1.36(qd, 2H), 1.40(m, 1H), 1.80(d, 2H), 1.90(t, 2H), 2.18(d, 2H), 2.38(s, 6H), 2.55(t, 2H), 3.40(t, 2H), 4.20(m, 1H), 5.64(d, 1H), 6.39(s, 1H), 8.41(s, 1H), 8.80(s, 1H).

MS (APCl): m/z 343 [MH]⁺.

### EXAMPLE 30

N*6*-(3-Dimethylamino-propyl)-N*4*-(4,4-dimethyl-cyclohexyl)-pyrido[3,4-d]Pyrimidine-4,6-diamine

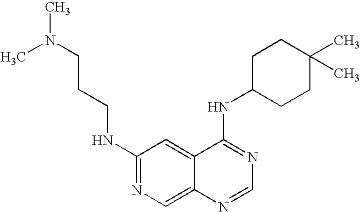

This compound was prepared by a method similar to that described for Example 24 using the fluoro compound of Preparation 10 and dimethyl-propane-1,3-diamine as starting materials. The title product was obtained as a yellow solid in 50% yield.

¹HNMR (MeOD, 400 MHz): 8.6 (s, 1H), 8.2 (s, 1H), 6.95 (s, 1H), 4.1 (s, 1H), 3.4 (t, 2H), 2.5 (t, 2H), 2.3 (s, 6H), 1.85 (m, 4H), 1.7 (m, 2H), 1.5 (m, 4H), 1.05 (s, 3H), 1.0 (s, 3H).

MS (APCl+) m/z 357 [MH]⁺.

### EXAMPLE 31

N*6*-(4-Dimethylamino-butyl)-N*4*-(4,4-dimethyl-cyclohexyl)-pyrido[3,4-d]pyrimidine-4,6-diamine

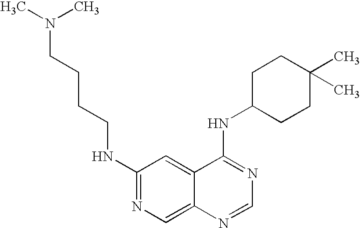

This compound was prepared by a method similar to that described for Example 24 using the fluoro compound of Preparation 10 and dimethyl-butane-1,4-diamine as starting materials. The title product was obtained as a yellow solid in 32% yield.

¹HNMR (MeOD, 400 MHz): 8.6 (s, 1H), 8.2 (s, 1H), 6.9 (s, 1H), 4.1 (s, 1H), 3.3 (t, 2H), 2.4 (t, 2H), 2.25 (s, 6H), 1.85 (m, 2H), 1.7 (m, 6H), 1.5 (m, 4H), 1.05 (s, 3H), 0.95 (s, 3H).

MS (APCl+) m/z 370 [MH]⁺.

### EXAMPLE 32

N*4*-Cycloheptyl-N*6*-[2-(1-methyl-pyrrolin-2-yl)-ethyl]-pyrido[3,4-d]pyrimidine-4,6-diamine

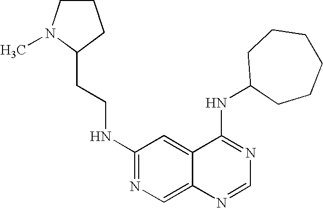

This compound was prepared by a method similar to that described for Example 24 using the fluoro compound of Preparation 1 and 2-(1-methyl-pyrrolidin-2-yl)-ethylamine as starting materials. The title product was obtained as a yellow solid in 35% yield.

¹HNMR (CDCl₃, 400 MHz): 8.8 (s, 1H), 8.45 (s, 1H), 6.2 (s, 1H), 5.6 (bs, 1H), 5.35 (bs, 1H), 4.2 (m, 1H), 3.4 (t, 2H), 3.2 (t, 1H), 2.4 (s, 3H), 2.3 (m, 1H), 2.1 (2H, m), 2.0 (m, 2H), 1.9-1.5 (m, 15H).

MS (APCl+) m/z 369 [MH]⁺.

### EXAMPLE 33

N*4*Cycloheptyl-N*6-(3-pyrrolidin-1-yl-propyl)-pyrido[3,4-d]pyrimidine-4.6-diamine

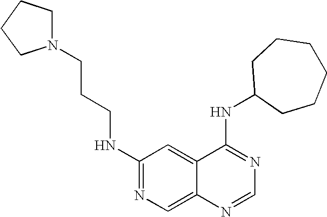

This compound was prepared by a method similar to that described for Example 24 using the fluoro compound of Preparation 1 and 3-pyrrolidin-1-yl-propylamine as starting materials. The title product was obtained as a yellow solid in 28% yield.

¹HNMR (CDCl₃, 400 MHz): 8.8 (s, 1H), 8.4 (s, 1H), 6.3 (s, 1H), 5.6 (bs, 1H), 5.3 (bs, 1H), 5.4 (m, 1H), 3.4 (t, 2H), 2.7 (m, 6H), 2.1 (m, 2H), 1.9 (m, 2H), 1.85 (m, 4H), 1.8-1.5 (m, 10H).

MS (APCl+) m/z 369 [MH]⁺.

### EXAMPLE 34

N*4-Cycloheptyl-N*6*-(4-dimethylamino-butyl)pyrido[3,4-d]pyrimidine-4,6-diamine

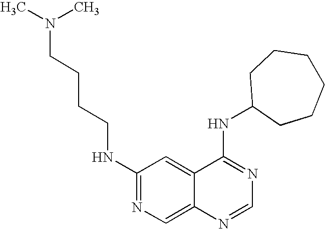

This compound was prepared by a method similar to that described for Example 24 using the fluoro compound of Preparation 1 and dimethyl-butane-1,4-diamine as starting materials. The title product was obtained as a yellow solid in 22% yield.

¹HNMR (MeOD, 400 MHz): 8.6 (s, 1H), 8.2 (s, 1H), 6.9 (s, 1H), 4.2 (m, 1H), 3.35 (t, 2H), 2.4 (t, 2H), 2.25 (s, 6H), 2.1 (m, 2H), 1.8-1.6 (m, 14H).

MS (APCl+) m/z 357 [MH]⁺.

### EXAMPLE 35

Cycloheptyl-[6-(1-methyl-piperidin-4-ylmethoxy)-pyrido[3,4-d]pyrimidin-4-yl]-amine

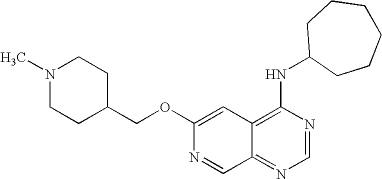

Sodium hydride (51 mg, 1.27 mmol) was added to a mixture of 4-(hydroxymethyl)-1-methylpiperidine hydroiodide (260 mg, 1.01 mmol) and 1-methyl-2-pyrrolidinone (1 ml) under nitrogen. The reaction mixture was stirred at room temperature for 5 minutes and then cycloheptyl-(6-fluoro-pyrido[3,4-d]pyrimidin-4-yl)-amine (88 mg, 0.34 mmol) was added and the resulting mixture was heated to 180° C. for 800s using microwave irradiation. The mixture was diluted with water (10 ml) and extracted with diethyl ether (3×10 ml). The combined organic extracts were loaded directly onto a silica gel column. Elution with dichloromethane:methanol:ammonia (95:4.5:0.5 by volume) yielded a pale yellow oil. The oil was crystallised from acetonitrile to yield 40 mg of title product (32%).

¹HNMR(d₆-DMSO): 1.39-2.01 (m, 19H), 2.13 (s, 3H), 2.76 (m 2H), 4.14 (m, 2H), 4.32 (m, 1H), 7.66 (s, 1H), 8.05 (d, 1H), 8.37 (s, 1H), 8.72 (s, 1H) MS (APCl+) m/z 370 [MH]⁺.

### EXAMPLE 36

Cycloheptyl-{6-[3-(4-methyl-piperazin-1-yl)-propoxy]-pyrido[3,4-d]pyrimidin-4-yl}-amine

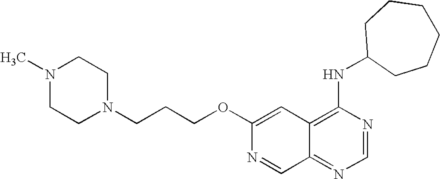

The title compound was prepared by a method similar to that described for Example 35 using the fluoro compound of Preparation 1 and 1-(3-hydroxypropyl)-4-methylpiperazine as starting materials. The title product was obtained as a white solid in 30% yield.

¹HNMR(d₆-DMSO): 1.40-1.71 (m, 15H), 2.12 (s, 3H), 2.21-2.43 (m 9H), 4.28-4.35 (m, 1H), 4.31 (t, 2H), 7.66 (s, 1H), 8.06 (d, 1H), 8.36 (s, 1H), 8.73 (s, 1H).

MS (APCl+) m/z 400 [MH]⁺.

### EXAMPLE 37

Cycloheptyl-{6-[2-(4-methyl-piperazin-1-yl)-ethoxy]-Pyrido[3,4-d]pyrimidin-4-yl}-amine

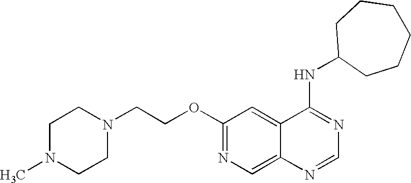

This compound was prepared by a method similar to that described for Example 35 using the fluoro compound of Preparation 1 and 1-(2-hydroxyethyl)-4-methylpiperazine as starting materials. The title product was obtained as a white solid in 19% yield.

¹H NMR(d₆-DMSO, 400 MHz) 1.41-1.72(m, 12H), 1.93(m, 2H), 2.12(s, 3H), 2.28(m, 4H), 2.48(m, 2H), 2.69 (t, 2H), 4.31(m, 1H), 4.39(t, 2H), 7.68(s, 1H), 8.05(d, 1H), 8.37(s, 1H).

MS (APCl+) m/z 385 [MH]⁺.

### EXAMPLE 38

N*4*-Cyclooctyl-N*6*-(3-dimethylamino-propyl)-pyrido[3,4-d]pyrimidine-4,6-diamine

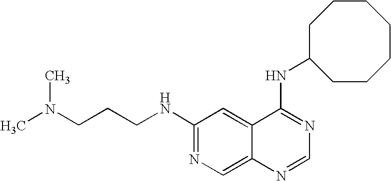

This compound was prepared by a method similar to that described for Example 24 using the fluoro compound of Preparation 11 and N,N-dimethylaminopropylamine as starting materials. The title product was obtained as a yellow solid in 37% yield.

¹HNMR(CDCl₃): 1.50-1.85(m, 14H), 2.00(m, 2H), 2.25(s, 6H), 2.40(t, 2H), 3.30(t, 2H), 4.40(m, 1H), 5.45(br s, 1H), 5.60(d, 1H), 6.15(s, 1H), 8.20(s, 1H), 8.80(s, 1H).

MS (APCl) m/z 357 [MH]⁺.

The following Preparations describe the synthesis of certain intermediates used in the preceding Examples.

Preparation 1

Cycloheptyl-(6-fluoro-pyrido[3,4-d]pyrimidin-4-yl)amine

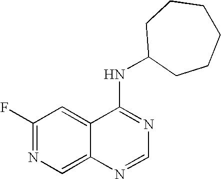

Cycloheptylamine (1.4 ml, 10.9 mmol) was added in 200 μl portions to a stirred solution of 4-chloro-6-fluoro-pyrido[3,4-d]pyrimidine (1.0g, 5.45 mmol) (J. Med. Chem., 1998, 41, 742) in dichloromethane (12 ml) over a period of 10 minutes. The reaction mixture was stirred at room temperature for 18 hours. The reaction mixture was partitioned between dichloromethane (100 ml) and an aqueous solution of acetic acid (pH3, 100 ml) and the organic layer was separated and diluted with methanol (3 ml) to bring all organic material into solution. The organic layer was dried over magnesium sulphate and concentrated in vacuo. The crude product was dissolved in dichloromethane and purified by column chromatography on silica gel eluting with dichloromethane:methanol 100:0 to 92:8 (by volume) to yield the title product as an off white solid (1.59g).

¹H NMR(CD₃OD, 400 MHz): 1.56-1.83(m, 10H), 1.98(m, 2H), 4.41(m, 1H), 7.82(m, 1H), 8.48(m, 1H), 8.74(m, 1H).

MS (ESI+) m/z 261 [MH]⁺.

Preparation 2

Cyclohexyl-(6-fluoro-pyrido[3,4-d]pyrimidin-4-yl)-amine

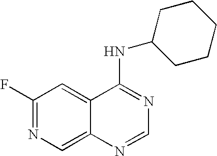

The title compound was prepared by a method similar to that described for Preparation 1 using cyclohexylamine as the relevant starting material. The title compound was isolated as an off white solid in 88% yield.

¹H NMR(CD₃OD, 400 MHz):1.27(m, 1H), 1.44(brm, 4H), 1.73(m, 1H), 1.84(m, 2H), 2.07(m, 2H), 4.11(m, 1H), 7.81(m, 1H), 8.46(m, 1H), 8.75(m, 1H).

MS (ESI+) m/z 247 [MH]⁺.

Preparation 3

(4,4-Difluoro-cyclohexyl)-(6-fluoro-pyrido[3,4-d]pyrimidin-4-yl)-amine

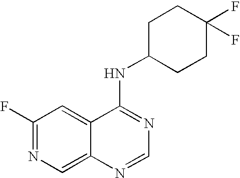

The title compound was prepared by a method similar to that described for Preparation 1 except that the reaction mixture was treated with N-ethyldiisopropylamine (3 equivalents) prior to addition of 4,4-difluorocyclohexylamine. Title product was isolated as a solid in 47% yield.

¹H NMR(CD₃OD, 400 MHz): 1.72-2.23(brm, 8H), 4.48(m, 1H), 7.79(m, 1H), 8.47(m, 1H), 8.78(m, 1H).

MS (ESI+) m/z 283 [MH]⁺.

Preparation 4

(6-Fluoro-Pyrido[3,4-d]pyrimidin-4-yl)indan-2-yl-amine

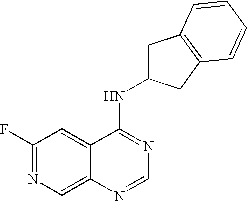

The title compound was prepared by a method similar to that described for Preparation 1 using indan-2-ylamine as the relevant starting material.

¹H NMR(CDCl₃, 400 MHz): 3.03(dd, 2H), 3.52(dd, 2H), 5.18(m, 1H), 6.13(brs, 1H), 7.14(s, 1H), 7.22(m, 2H), 7.28(m, 2H), 8.73(m, 1H), 8.96(m, 1H).

Preparation 5

N*6*-(2-Amino-ethyl)-N*4*-cyclohexyl-pyrido[3,4-d]pyrimidine-4,6-diamine

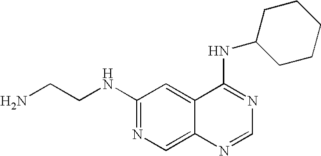

The fluoro compound of Preparation 2 (600 mg, 2.44 mmol) was added to ethane-1,2-diamine (4.13 ml, 60.9 mmol) and the reaction mixture was heated to 130° C. for 3 hours. The reaction mixture was concentrated in vacuo and the crude product was purified by column chromatography on silica gel eluting with dichloromethane:methanol:ammonia 100:0:0 to 85:15:2 (by volume) to yield the title product as a yellow oil (653 mg, 93% yield).

¹H NMR(CD₃OD, 400 MHz): 1.26(m, 1H), 1.44(m, 4H), 1.72(m, 1H), 1.86(m, 2H), 2.08(m, 2H), 2.89(m, 2H), 3.41(m, 2H), 4.16(m, 1H), 6.91(m, 1H), 8.18(m, 1H), 8.60(m, 1H).

MS (ESI+) m/z 287 [MH]⁺.

Preparation 6

(6-Fluoro-pyrido[3,4-d]pyrimidin-4-yl)-(tetrahydro-furan-2-ylmethyl)-amine

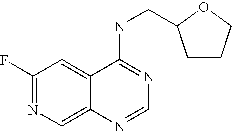

Tetrahydrofurfurylamine (1.35 ml, 13.1 mmol) was added to a stirred solution of 4-chloro-6-fluoro-pyrido[3,4-d]pyrimidine (2g, 10.9 mmol) and diisopropylethylamine (2.8 ml, 16.3 mmol) in dichloromethane (10 ml). The reaction mixture was stirred at room temperature for 18 hours and then partitioned between water and dichloromethane. The organic layer was concentrated in vacuo to yield an off white solid which was recrystallised from acetonitrile to yield the title product as a white solid (2.3g, 85%).

¹H NMR(CD₃OD, 400 MHz): 1.70(m, 1H), 1.95(m, 2H), 2.07(m, 1H), 3.66(dd, 1H), 3.78(m, 2H), 3.91(dd, 1H), 4.25(m, 1H), 7.73(s, 1H), 8.50(s, 1H), 8.76(s, 1H).

Preparation 7

(6-Fluoro-pyrido[3,4-d]pyrimidin-4-yl)-pyridin-2-ylmethyl-amine 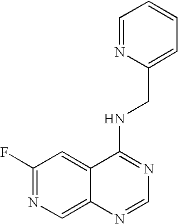
2-Methylaminopyridine (4.3 ml, 42 mmol) was added to a stirred solution of 4-chloro-6-fluoro-pyrido[3,4-d]pyrimidine (6.1 g, 33.3 mmol) and diisopropylethylamine (14.5 ml, 83.3 mmol) in dichloromethane (250 ml). The reaction mixture was stirred at room temperature for 16 hours and washed with water (3×200 ml) and brine (200 ml). The organic phase was dried (Na₂SO₄) and concentrated under vacuo to yield an off white solid which was recrystallised in acetonitrile to yield the title product as a white solid (2.4 g, 28%).

¹H NMR(CDCl₃, 400 MHz): 4.92(s, 2H), 7.31(dd, 1H), 7.36-7.41(m, 2H), 7.77(td, 1H), 7.90(br s, 1H), 8.64(d, 1H), 8.71 (s, 1H), 8.95(s, 1H).

MS (APCl+) m/z 256 [MH]⁺.

Preparation 8

(6-Fluoro-pyrido[3,4-d]pyrimidin-4-yl)-(6-methyl-pyridin-2-ylmethyl)-amine 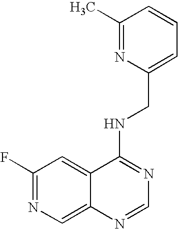
2-Methylamino-6-methylpyridinium hydrochloride (2.5g, 16. mmol) was added to a stirred solution of 4-chloro-6-fluoro-pyrido[3,4-d]pyrimidine (2.45g, 13.3 mmol) and diisopropylethylamine (12 ml, 66. mmol) in dichloromethane (250 ml). The reaction mixture was stirred at room temperature for 16 hours and then washed with water (2×150 ml) and brine (150 ml). The organic phase was dried (Na₂SO₄) and concentrated under vacuo to yield a purple solid which was recrystallised in acetonitrile to yield the title product as a pale solid, (2.1g, 59%).

¹H NMR(d₆-DMSO, 400 MHz): 2.46(s, 3H), 4.81(s, 2H), 7.12(t, 2H), 7.58(t, 1H), 8.03(s, 1H), 8.50(s, 1H), 8.83(s, 1H), 9.24(br s, 1H).

MS (APCl+) m/z 270 [MH]⁺.

Preparation 9

(6-Fluoro-pyrido[3,4-d]pyrimidin-4-yl)-(trans-4-methyl-cyclohexyl)-amine 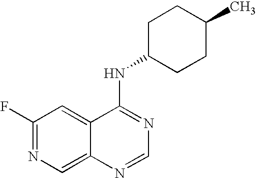
4-Chloro-6-fluoro-pyrido[3,4-d]pyrimidine (7.14g, 38.8 mmol) was added to a stirred solution of trans-4-methylcyclohexylamine (4.0g, 35.3 mmol, see Journal of Organic Chemistry 2000, 65(21), 7098-7104) and diisopropylethylamine (12.3 ml, 70.6 mmol) in dichloromethane (150 ml) at 0° C. The reaction mixture was allowed to warm to room temperature and stirred for 16 hours. Water (50 ml) was added and the mixture was stirred for 2 hours. The layers were separated and the organic layer was washed with water (2×50 ml) and concentrated under vacuo to yield an off white solid. The solid was purified by silica gel chromatography (eluting with dichloromethane and then 1:2 ethyl acetate:dichloromethane) and the resulting solid was recrystallised from toluene to afford the title compound as a white crystalline solid (2.35g, 26%).

Melting point 169-171° C.

¹H NMR(CDCl₃, 400 MHz): 0.96(d, 3H), 1.17(qd, 2H), 1.28(qd, 2H), 1.43(m, 1H), 1.82(dd, 2H), 2.18(dd, 2H), 4.21(m, 1H), 5.76(br s, 1H), 7.18(s, 1H), 8.68(s, 1H), 8.94(s, 1H).

MS (APCl): m/z 261 [MH]⁺.

Preparation 10

(4,4-Dimethyl-cyclohexyl)-(6-fluoro-pyrido[3,4-d]pyrimidin-4-yl)-amine 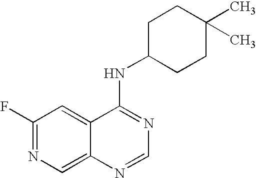
4,4-Dimethyl-cyclohexylamine (380 mg, 2.3 mmol, see Journal of Medicinal Chemistry, 1971, 14, 600-614) was added to a stirred solution of 4-chloro-6-fluoro-pyrido[3,4-d]pyrimidine (500 mg, 2.7 mmol) and triethylamine (75 μL, 5.4 mmol) in dichloromethane (10 ml). The reaction mixture was stirred at room temperature for 18 hours. The reaction mixture was partitioned between water and dichloromethane. The organic layer was concentrated in vacuo. The resultant solid was purified by column chromatography to give the title compound as a white solid (450 mg, 69%).

¹HNMR (CDCl₃, 400 MHz): 8.95(s, 1H), 8.65(s, 1H), 7.15(s, 1H), 5.7(bs, 1H), 4.2(m, 1H), 2.0(m, 2H), 1.6-1.4(m, 6H), 1.95(s, 6H).

MS (APCl+) m/z 275 [MH]⁺.

Preparation 11

Cyclooctyl-(6-fluoro-pyrido[3,4-d]pyrimidin-4-yl)-amine

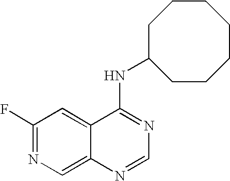

Cyclooctylamine (692 mg, 5.4 mmol) was added to a stirred solution of 4-chloro-6-fluoro-pyrido[3,4-d]pyrimidine (0.5g, 2.7 mmol) and diisopropylethylamine (0.95 ml, 5.4 mmol) in dichloromethane (13 ml) at 5° C. The reaction mixture was then stirred at room temperature for 18 hours. The reaction mixture was partitioned between water and dichloromethane. The organic layer was concentrated in vacuo to yield an off white solid which was recrystallised in acetonitrile to yield the title compound as a tan solid (0.2g, 27%).

¹H NMR(CDCl₃, 400 MHz): 1.55-1.80(m, 12H), 2.00(m, 2H), 4.50(m, 1H), 5.70(brs, 1H), 7.10(s, 1H), 8.65(s, 1H), 8.95(s, 1H).

MS (APCl+) m/z 275 [MH]⁺.

1. A compound of formula (I): 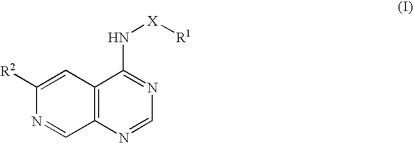
or a pharmaceutically acceptable salt or solvate thereof, wherein
X is a bond or C₁-C₃ alkylene;
R¹ is (a) C₃-C₈ cycloalkyl optionally substituted with one or more substituents selected from halo, oxo, C₁-C₆ alkyl, C₃-C₈ cycloalkyl, Het¹, C₁-C₆ alkoxy and cyano, wherein one or two of the methylene (—CH₂—) groups of said C₃-C₈ cycloalkyl may optionally be replaced by an —NR³—, —O— or —S(O)ₙ— group and wherein said C₃-C₈ cycloalkyl, whether modified as indicated above or not, may be optionally benzo-fused, said benzo-fused portion being optionally substituted by one or more substituents selected from halo, C₁-C₆ alkyl, C₃-C₈ cycloalkyl, C₁-C₆ alkoxy and cyano; or
(b) C₃-C₈ cycloalkyl spiro fused to a C₃-C₈ cycloalkyl or Het¹ group; or
(c) Het²;
with the proviso that R¹ may not be Het² when X is a bond;
R is —OR⁴ or —NR⁴R⁵;
R³ is H, C₁-C₆ alkyl or C₃-C₈ cycloalkyl;
R⁴ is C₁-C₆ alkyl or C₃-C₈ cycloalkyl, said C₁-C₆ alkyl and C₃-C₈ cycloalkyl being optionally substituted by one or more R⁶ or —(C₁-C₆ alkylene)-R⁶ groups and optionally having one methylene group (—CH₂—) replaced by an —NR³—, —O— or —S(O)ₙ— group and said C₃-C₈ cycloalkyl, whether modified as indicated above or not, being optionally benzo-fused, said benzo-fused portion being optionally substituted by one or more substituents selected from halo, C₁-C₆ alkyl, C₃-C₈ cycloalkyl, C₁-C₆ alkoxy and cyano; and
R⁵ is H, C₁-C₆ alkyl or C₃-C₈ cycloalkyl;
or, in the case where R² is —NR⁴R⁵, R⁴ and R⁵, taken together, with the nitrogen atom to which they are attached, form a saturated heterocyclic group selected from aziridinyl, azetidinyl, pyrrolidinyl, piperidinyl, morpholinyl, thiomorpholinyl, piperazinyl, azepinyl or diazapinyl, wherein said heterocyclic group is optionally substituted on a ring carbon atom by one or more R⁶ or —(C₁-C₆ alkylene)-R⁶ groups, optionally substituted on a ring nitrogen atom by one or more R⁹ groups and optionally benzo-fused, said benzo-fused portion being optionally substituted by one or more substituents selected from halo, C₁-C₆ alkyl, C₃-C₈ cycloalkyl, C₁-C₆ alkoxy and cyano;
R⁶ is Het¹, Het², —OR⁷—SR⁷, —SOR⁸, —SO₂R⁸, —NR⁷R⁷, —COR⁷, —OCOR⁷, —SCOR⁷,
—NR⁷COR⁷, —NR⁷SO₂R⁸, —COOR⁷, —COSR⁷, —CONR⁷R⁷, —OCOOR⁸, OCOSR⁸,
—OCONR⁷R⁷, —NR⁷COOR⁷, —NR⁷COSR⁷, —NR⁷CONR⁷R⁷, oxo, halo, —CN, C₁-C₆ alkyl, C₃-C₈ cycloalkyl, C₂-C₆ alkenyl, C₂-C₆ alkynyl or aryl;
each R⁷ is independently selected from H, C₁-C₆ alkyl and C₃-C₈ cycloalkyl;
each R⁸ is independently selected from C₁-C₆ alkyl and C₃-C₈ cycloalkyl;
R⁹ is C-linked Het¹, C-linked Het², —SO₂R⁸, —COR⁷, —COOR⁸, —COSR⁸, —CONR⁷R⁷, C₁-C₆ alkyl, C₃-C₈ cycloalkyl, C₂-C₆ alkenyl, C₂-C₆ alkynyl or aryl;
n is 0, 1 or 2;
Het¹ is a 3- to 8-membered, saturated or partially unsaturated heterocyclic group comprising one or two ring members selected from —NR¹⁰—, —O— and —S(O)ₙ—, said heterocyclic group being optionally substituted on a ring carbon atom by one or more substituents selected from oxo, halo, —R⁸ or —OR⁸ and optionally benzo-fused, said benzo-fused portion being optionally substituted by one or more substituents selected from halo, C₁-C₆ alkyl, C₃-C₈ cycloalkyl, C₁-C₆ alkoxy and cyano;
R¹⁰ is H, C₁-C₆ alkyl or C₃-C₈ cycloalkyl, —COR⁸, —SO₂R⁸ or a bond to the group which is substituted with Het¹;
Het² is a 5-membered aromatic heterocyclic group comprising either (a) 1 to 4 nitrogen atoms, (b) one oxygen or one sulphur atom or (c) 1 oxygen atom or 1 sulphur atom and 1 or 2 nitrogen atoms or a 6-membered aromatic heterocyclic group comprising 1 or 2 nitrogen atoms, said 5- or 6-membered heterocyclic group being optionally substituted by one or more substituents selected from halo, —NR⁷R⁷, C₁-C₆ alkyl, C₃-C₈ cycloalkyl, C₁-C₆ alkoxy and cyano and optionally benzo-fused, said benzo-fused portion being optionally substituted by one or more substituents selected from halo, —NR⁷R⁷, C₁-C₆ alkyl, C₃-C₈ cycloalkyl, C₁-C₆ alkoxy and cyano; and
aryl is phenyl or naphthyl optionally substituted by one or more substituents selected from halo, —NR⁷R⁷, C₁-C₆ alkyl, C₃-C₈ cycloalkyl, C₁-C₆ alkoxy and cyano.

2. A compound of formula (I), or a pharmaceutically acceptable salt or solvate thereof, as claimed in claim 1, wherein X is methylene or a bond.

3. A compound of formula (I), or a pharmaceutically acceptable salt or solvate thereof, as claimed in claim 1, wherein R¹ is (i) C₅-C₈ cycloalkyl optionally substituted with one or more substituents selected from halo and C₁-C₆ alkyl, wherein one of the methylene (—CH₂—) groups of said C₅-C₈ cycloalkyl may optionally be replaced by an —O— group and wherein said C₅-C₈ cycloalkyl, whether modified as indicated above or not, may be optionally benzo-fused; or (ii) pyridyl optionally substituted by one or more C₁-C₆ alkyl groups; with the proviso that R¹ may not be optionally substituted pyridyl when X is a bond.

4. A compound of formula (I), or a pharmaceutically acceptable salt or solvate thereof, as claimed in claim 1, wherein R² is —OR⁴ or —NR⁴R⁵ and R⁴ is C₁-C₅ alkyl or C₅-C₆ cycloalkyl, said C₁-C₅ alkyl and C₅-C₆ cycloalkyl being optionally substituted by one or more groups selected from Het¹, Het², —OR⁷—NR⁷R⁷ and —NR⁷SO₂R⁷ and optionally having one methylene (—CH₂—) group replaced by an —O— group and said C₅-C₆ cycloalkyl, whether modified as indicated above or not, being optionally benzo-fused and R⁵ is H; or in the case where R² is —NR⁴R⁵, R⁴ and R⁵ when taken together with the nitrogen atom to which they are attached, form a saturated heterocylic group selected from azetidinyl, piperdinyl and piperazinyl, wherein said heterocyclic group is optionally substituted on a ring carbon atom by one ore more groups selected from —OR⁷—(C₁-C₆ alkylene)-NR⁷R⁷ and halo and optionally substituted on a ring nitrogen atom by —COR⁷.

5. A compound of formula (I), as claimed in claim 1, which is:
2-{2-[4-(indan-2-ylamino)-pyrido[3,4-d]pyrimidin-6-ylamino]-ethoxy}-ethanol;
N*4*-cycloheptyl-N*6*-(3-dimethylamino-propyl)-pyrido[3,4-d]pyrimidine-4,6-diamine; or
cycloheptyl-[6-(2-morpholin-4-yl-ethoxy)-pyrido[3,4-d]pyrimidin-4-yl]-amine;
or a pharmaceutically acceptable salt or solvate thereof.

6. A pharmaceutical formulation including a compound of formula (I), or a pharmaceutically acceptable salt or solvate thereof, as claimed in claim 1 and a pharmaceutically acceptable excipient.

7. A method of treating pain in a mammal, including a human being, including administering to said mammal an effective amount of a compound of formula (I), or a pharmaceutically acceptable salt or solvate thereof, as claimed in claim 1.

8. A combination of a compound of formula (I), or a pharmaceutically acceptable salt or solvate thereof, as claimed in claim 1 and a second pharmacologically active compound.

9. A compound of formula (I), or a pharmaceutically acceptable salt or solvate thereof, as claimed in claim 1, wherein:
X is a bond or C₁-C₃ alkylene;
R¹ is (a) C₃-C₈ cycloalkyl optionally substituted with one or more substituents selected from halo, oxo, C₁-C₆ alkyl, C₁-C₆ alkoxy and cyano, wherein one or two of the methylene (—CH₂—) groups of said C₃-C₈ cycloalkyl may optionally be replaced by an —NR³—, —O— or —S(O)ₙ— group and wherein said C₃-C₈ cycloalkyl, whether modified as indicated above or not, may be optionally benzo-fused, said benzo-fused portion being optionally substituted by one or more substituents selected from halo, C₁-C₆ alkyl, C₃-C₈ cycloalkyl, C₁-C₆ alkoxy and cyano; or
(b) Het²;
with the proviso that R¹ may not be Het² when X is a bond;
R² is —OR⁴ or —NR⁴R⁵;
R³ is H, C₁-C₆ alkyl or C₃-C₈ cycloalkyl;
R⁴ is C₁-C₆ alkyl or C₃-C₈ cycloalkyl, said C₁-C₆ alkyl and C₃-C₈ cycloalkyl being optionally substituted by one or more R⁶ groups and optionally having one methylene group (—CH₂—) replaced by an —NR³—, —O— or —S(O)ₙ-group and said C₃-C₈ cycloalkyl, whether modified as indicated above or not, being optionally benzo-fused, said benzo-fused portion being optionally substituted by one or more substituents selected from halo, C₁-C₆ alkyl, C₃-C₈ cycloalkyl, C₁-C₆ alkoxy and cyano; and
R⁵ is H, C₁-C₆ alkyl or C₃-C₈ cycloalkyl;
or, in the case where R² is —NR⁴R⁵, R⁴ and R⁵, taken together, with the nitrogen atom to which they are attached, form a saturated heterocyclic group selected from aziridinyl, azetidinyl, pyrrolidinyl, piperidinyl, morpholinyl, thiomorpholinyl, piperazinyl, azepinyl or diazapinyl, wherein said heterocyclic group is optionally substituted on a ring carbon atom by one or more R⁶ groups, optionally substituted on a ring nitrogen atom by one or more R⁹ groups and optionally benzo-fused, said benzo-fused portion being optionally substituted by one or more substituents selected from halo, C₁-C₆ alkyl, C₃-C₈ cycloalkyl, C₁-C₆ alkoxy and cyano;
R⁶ is Het¹, Het², —OR⁷, —SR⁷, —SOR⁸, —SO₂R⁸, —NR⁷R⁷, —COR⁷, —OCOR⁷, —SCOR⁷,
—NR⁷COR⁷, —NR⁷SO₂R⁸, —COOR⁷, —COSR⁷, —CONR⁷R⁷, —OCOOR⁸, OCOSR⁸,
—OCONR⁷R⁷, —NR⁷COOR⁷, —NR⁷COSR⁷, —NR⁷CONR⁷R⁷, oxo, halo, —CN, C₁-C₆ alkyl, C₃-C₈ cycloalkyl, C₂-C₆ alkenyl, C₂-C₆ alkynyl or aryl;
each R⁷ is independently selected from H, C₁-C₆ alkyl and C₃-C₈ cycloalkyl;
each R⁸ is independently selected from C₁-C₆ alkyl and C₃-C₈ cycloalkyl;
R⁹ is C-linked Het¹, C-linked Het², —SO₂R⁸, —COR⁷, —COOR⁸, —COSR⁸, —CONR⁷R⁷, C₁-C₆ alkyl, C₃-C₈ cycloalkyl, C₂-C₆ alkenyl, C₂-C₆ alkynyl or aryl;
n is 0, 1 or 2;
Het¹ is a 3- to 8-membered, saturated or partially unsaturated heterocyclic group comprising one or two ring members selected from —NR¹⁰—, —O— and —S(O)ₙ—, said heterocyclic group being optionally substituted on a ring carbon atom by one or more substituents selected from oxo, halo, —R⁸ or —OR⁸ and optionally benzo-fused, said benzo-fused portion being optionally substituted by one or more substituents selected from halo, C₁-C₆ alkyl, C₃-C₈ cycloalkyl, C₁-C₆ alkoxy and cyano;
R¹⁰ is H, C₁-C₆ alkyl or C₃-C₈ cycloalkyl, —COR⁸, —SO₂R⁸ or a bond to the group which is substituted with Het¹;
Het² is a 5-membered aromatic heterocyclic group comprising either (a) 1 to 4 nitrogen atoms, (b) one oxygen or one sulphur atom or (c) 1 oxygen atom or 1 sulphur atom and 1 or 2 nitrogen atoms or a 6-membered aromatic heterocyclic group comprising 1 or 2 nitrogen atoms, said 5- or 6-membered heterocyclic group being optionally substituted by one or more substituents selected from halo, —NR⁷R⁷, C₁-C₆ alkyl, C₃-C₈ cycloalkyl, C₁-C₆ alkoxy and cyano and optionally benzo-fused, said benzo-fused portion being optionally substituted by one or more substituents selected from halo, —NR⁷R⁷, C₁-C₆ alkyl, C₃-C₈ cycloalkyl, C₁-C₆ alkoxy and cyano; and
aryl is phenyl or naphthyl optionally substituted by one or more substituents selected from halo, —NR⁷R⁷, C₁-C₆ alkyl C₃-C₈ cycloalkyl, C₁-C₆ alkoxy and cyano.

10. A compound of formula (II): 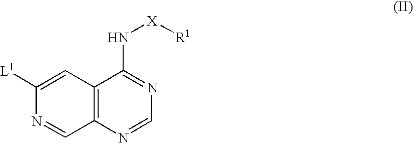
wherein L¹ is a suitable leaving group and X and R¹ are as defined in claim 1.
